(19) United States
(12) Patent Application Publication
(10) Pub. No.: US 2007/0179614 A1
(43) Pub. Date: Aug. 2, 2007

(54) Intervertebral prosthetic disc and method of installing same

(75) Inventors: Eric S. Heinz, Memphis, TN (US); Jason Eckhardt, Memphis, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/343,081

(22) Filed: Jan. 30, 2006

(51) Int. Cl. A61F 2/44

(52) U.S. Cl. 623/17.12; 623/17.14

(57) ABSTRACT

An intervertebral prosthetic disc is disclosed. The intervertebral prosthetic disc can be installed within an intervertebral space between adjacent first and second vertebrae. The intervertebral prosthetic disc can include a first component that is configured to engage the first vertebra, a second component that is configured to engage the second vertebra, and at least one expandable motion limiter. Further, the expandable motion limiter can be configured to move from a deflated position to at least one inflated position.

### FIELD OF THE DISCLOSURE

The present disclosure relates generally to orthopedics and spinal surgery. More specifically, the present disclosure relates to intervertebral prosthetic discs.

### BACKGROUND

In human anatomy, the spine is a generally flexible column that can take tensile and compressive loads. The spine also allows bending motion and provides a place of attachment for ribs, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of deterioration.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan-view of a vertebra;

FIG. 4 is an anterior view of a first embodiment of an intervertebral prosthetic disc;

FIG. 5 is an exploded anterior view of the first embodiment of the intervertebral prosthetic disc;

FIG. 6 is a lateral view of the first embodiment of the intervertebral prosthetic disc;

FIG. 7 is an exploded lateral view of the first embodiment of the intervertebral prosthetic disc;

FIG. 8 is a plan view of a superior half of the first embodiment of the intervertebral prosthetic disc;

FIG. 9 is a plan view of an inferior half of the first embodiment of the intervertebral prosthetic disc;

FIG. 10 is an exploded lateral view of the first embodiment of the intervertebral prosthetic disc installed within an intervertebral space between a pair of adjacent vertrebrae;

FIG. 11 is an anterior view of the first embodiment of the intervertebral prosthetic disc installed within an intervertebral space between a pair of adjacent vertrebrae;

FIG. 12 is a lateral view of the first embodiment of the intervertebral prosthetic disc installed within an intervertebral space between a pair of adjacent vertrebrae;

FIG. 13 is a flow chart of a method of installing an intervertebral prosthetic disc within an intervertebral space between a pair of adjacent vertebrae;

FIG. 14 is an anterior view of a second embodiment of an intervertebral prosthetic disc;

FIG. 15 is an exploded anterior view of the second embodiment of the intervertebral prosthetic disc;

FIG. 16 is a lateral view of the second embodiment of the intervertebral prosthetic disc;

FIG. 17 is an exploded lateral view of the second embodiment of the intervertebral prosthetic disc;

FIG. 18 is a plan view of a superior half of the second embodiment of the intervertebral prosthetic disc;

FIG. 19 is a plan view of an inferior half of the second embodiment of the intervertebral prosthetic disc;

FIG. 20 is an anterior view of a third embodiment of an intervertebral prosthetic disc;

FIG. 21 is an exploded anterior view of the third embodiment of the intervertebral prosthetic disc;

FIG. 22 is a lateral view of the third embodiment of the intervertebral prosthetic disc;

FIG. 23 is an exploded lateral view of the third embodiment of the intervertebral prosthetic disc;

FIG. 24 is a plan view of a superior half of the third embodiment of the intervertebral prosthetic disc;

FIG. 25 is a plan view of an inferior half of the third embodiment of the intervertebral prosthetic disc;

FIG. 26 is an anterior view of a fourth embodiment of an intervertebral prosthetic disc;

FIG. 27 is an exploded anterior view of the fourth embodiment of the intervertebral prosthetic disc;

FIG. 28 is a lateral view of the fourth embodiment of the intervertebral prosthetic disc;

FIG. 29 is an exploded lateral view of the fourth embodiment of the intervertebral prosthetic disc;

FIG. 30 is a plan view of a superior half of the fourth embodiment of the intervertebral prosthetic disc;

FIG. 31 is a plan view of an inferior half of the fourth embodiment of the intervertebral prosthetic disc;

FIG. 32 is an anterior view of a fifth embodiment of an intervertebral prosthetic disc;

FIG. 33 is an exploded anterior view of the fifth embodiment of the intervertebral prosthetic disc;

FIG. 34 is a lateral view of the fifth embodiment of the intervertebral prosthetic disc;

FIG. 35 is an exploded lateral view of the fifth embodiment of the intervertebral prosthetic disc;

FIG. 36 is a plan view of a superior half of the fifth embodiment of the intervertebral prosthetic disc;

FIG. 37 is a plan view of an inferior half of the fifth embodiment of the intervertebral prosthetic disc;

FIG. 38 is an anterior view of a sixth embodiment of an intervertebral prosthetic disc;

FIG. 39 is an exploded anterior view of the sixth embodiment of the intervertebral prosthetic disc;

FIG. 40 is a lateral view of the sixth embodiment of the intervertebral prosthetic disc;

FIG. 41 is an exploded lateral view of the sixth embodiment of the intervertebral prosthetic disc;

FIG. 42 is a plan view of a superior half of the sixth embodiment of the intervertebral prosthetic disc;

FIG. 43 is a plan view of an inferior half of the sixth embodiment of the intervertebral prosthetic disc;

FIG. 44 is a lateral plan view of a first embodiment of an intervertebral prosthetic disc inserter;

FIG. 45 is an anterior plan view of the first embodiment of the intervertebral prosthetic disc inserter;

FIG. 46 is a top plan view of the first embodiment of the intervertebral prosthetic disc inserter;

FIG. 47 is a posterior plan view of the first embodiment of the intervertebral prosthetic disc inserter with a plunger removed;

FIG. 48 is a plan view of a stop cock;

FIG. 49 is a lateral plan view of a second embodiment of an intervertebral prosthetic disc inserter;

FIG. 50 is an anterior plan view of the second embodiment of the intervertebral prosthetic disc inserter;

FIG. 51 is a top plan view of the second embodiment of the intervertebral prosthetic disc inserter;

FIG. 52 is a plan view of a stop cock;

FIG. 53 is a lateral plan view of a third embodiment of an intervertebral prosthetic disc inserter;

FIG. 54 is an anterior plan view of the third embodiment of the intervertebral prosthetic disc inserter;

FIG. 55 is a top plan view of the third embodiment of the intervertebral prosthetic disc inserter;

FIG. 56 is an anterior plan view of the third embodiment of the intervertebral prosthetic disc inserter with the plungers removed;

FIG. 57 is an anterior plan view of the third embodiment of the intervertebral prosthetic disc inserter;

FIG. 58 is a lateral plan view of the fourth embodiment of the intervertebral prosthetic disc inserter;

FIG. 59 is an anterior plan view of the fourth embodiment of the intervertebral prosthetic disc inserter; and

FIG. 60 is a top plan view of the fourth embodiment of the intervertebral prosthetic disc inserter.

### DETAILED DESCRIPTION OF THE DRAWINGS

An intervertebral prosthetic disc is disclosed. The intervertebral prosthetic disc can be installed within an intervertebral space between adjacent first and second vertebrae. The intervertebral prosthetic disc can include a first component that is configured to engage the first vertebra, a second component that is configured to engage the second vertebra, and at least one expandable motion limiter. Further, the expandable motion limiter can be configured to move from a deflated position to at least one inflated position.

In another embodiment, an intervertebral prosthetic disc is disclosed and can be installed within an intervertebral space between an inferior vertebra and a superior vertebra. In this embodiment, the intervertebral prosthetic disc can include an inferior component that is configured to engage the inferior vertebra and a superior component that is configured to engage the superior vertebra. The superior component can include a superior support plate and at least one expandable motion limiter that can be attached to the superior support plate. The expandable motion limiter can be configured to move from a deflated position to one of a plurality of inflated positions.

In yet another embodiment an intervertebral prosthetic disc is disclosed and can be installed within an intervertebral space between an inferior vertebra and a superior vertebra. In this embodiment, the intervertebral prosthetic disc can include a superior component configured to engage the inferior vertebra and an inferior component configured to engage the inferior vertebra. Also, the inferior component can include an inferior support plate and at least one expandable motion limiter that can be attached to the inferior support plate. The expandable motion limiter can be configured to move from a deflated position to one of a plurality of inflated positions.

In yet another embodiment an intervertebral prosthetic disc is disclosed and can be installed within an intervertebral space between an inferior vertebra and a superior vertebra. In this embodiment, the intervertebral prosthetic disc can include a superior component configured to engage the superior vertebra and an inferior component configured to engage the inferior vertebra. Also, the superior component can include a superior support plate and at least one expandable motion limiter that can be attached to the superior support plate. The inferior component can include an inferior support plate and at least one expandable motion limiter that can be attached to the inferior support plate in a position(s) which would not contact or otherwise interfere with the motion limiter(s) of the superior plate through its/their full range of motion. The expandable motion limiter can be configured to move from a deflated position to one of a plurality of inflated positions.

In still another embodiment, a method of installing an intervertebral prosthetic disc within an intervertebral space between an inferior vertebra and a superior vertebra of a patient is disclosed. The method can include implanting the intervertebral prosthetic disc within the intervertebral space and inflating at least one expandable motion limiter within the intervertebral prosthetic disc.

Description of Relevant Anatomy

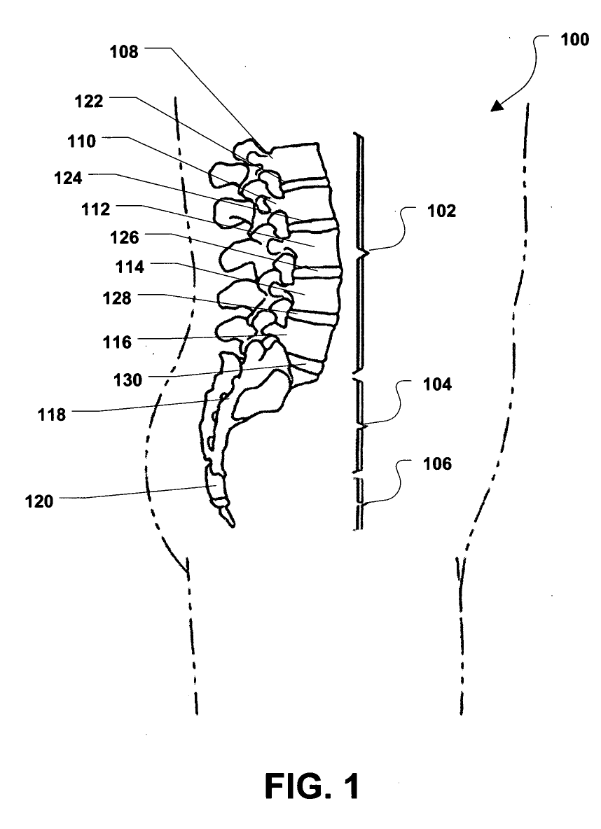

Referring initially to FIG. 1, a portion of a vertebral column, designated 100, is shown. As depicted, the vertebral column 100 includes a lumber region 102, a sacral region 104, and a coccygeal region 106. As is known in the art, the vertebral column 100 also includes a cervical region and a thoracic region. For clarity and ease of discussion, the cervical region and the thoracic region are not illustrated:

As shown in FIG. 1, the lumbar region 102 includes a first lumber vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumber vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

In a particular embodiment, if one of the intervertebral lumbar discs 122, 124, 126, 128, 130 is diseased, degenerated, damaged, or otherwise in need of replacement, that intervertebral lumbar disc 122, 124, 126, 128, 130 can be at least partially removed and replaced with an intervertebral prosthetic disc according to one or more of the embodiments described herein. In a particular embodiment, a portion of the intervertebral lumbar disc 122, 124, 126, 128, 130 can be removed via a discectomy, or a similar surgical procedure, well known in the art. Further, removal of intervertebral lumbar disc material can result in the formation of an intervertebral space (not shown) between two adjacent lumbar vertebrae.

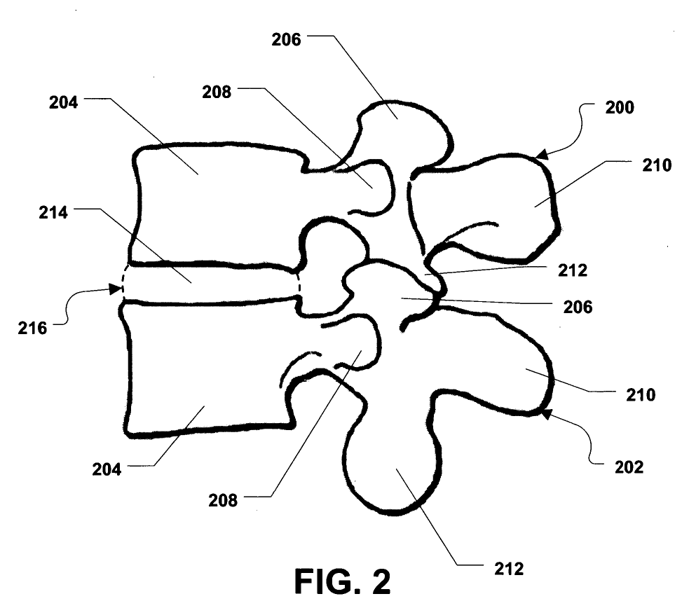

FIG. 2 depicts a detailed lateral view of two adjacent-vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines). As described in greater detail below, an intervertebral prosthetic disc according to one or more of the embodiments described herein can be installed within the intervertebral space 212 between the superior vertebra 200 and the inferior vertebra 202.

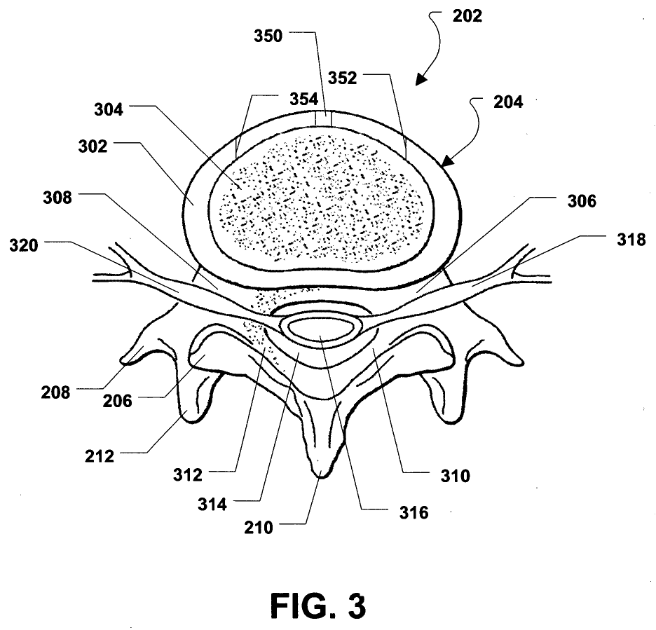

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

FIG. 3 further depicts a keel groove 350 that can be established within the cortical rim 302 of the inferior vertebra 202. Further, a first corner cut 352 and a second corner cut 354 can be established within the cortical rim 302 of the inferior vertebra 202. In a particular embodiment, the keel groove 350 and the corner cuts 352, 354 can be established during surgery to install an intervertebral prosthetic disc according to one or more of the embodiments described herein. The keel groove 350 can be established using a keel cutting device, e.g., a keel chisel designed to cut a groove in a vertebra, prior to the installation of the intervertebral prosthetic disc. Further, the keel groove 350 is sized and shaped to receive and engage a keel, described in detail below, that extends from an intervertebral prosthetic disc according to one or more of the embodiments described herein. The keel groove 350 can cooperate with a keel to facilitate proper alignment of an intervertebral prosthetic disc within an intervertebral space between an inferior vertebra and a superior vertebra.

### Description of a First Embodiment of an Intervertebral Prosthetic Disc

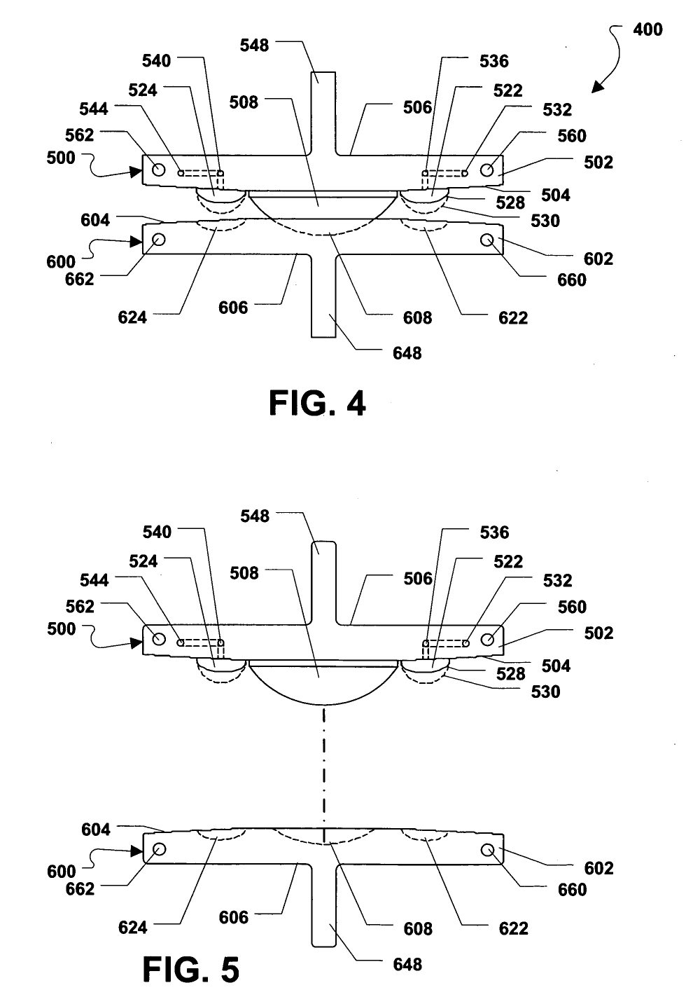

Referring to FIGS. 4 through 9 a first embodiment of an intervertebral prosthetic disc is shown and is generally designated 400. As illustrated, the intervertebral prosthetic disc 400 includes a superior component 500 and an inferior component 600. In a particular embodiment, the components 500, 600 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 500, 600 can be made from any other substantially rigid biocompatible materials.

In a particular embodiment, the superior component 500 includes a superior support plate 502 that has a superior articular surface 504 and a superior bearing surface 506. In a particular embodiment, the superior articular surface 504 can be generally curved and the superior bearing surface 506 can be substantially flat. In an alternative embodiment, the superior articular surface 504 can be substantially flat and at least a portion of the superior bearing surface 506 can be generally curved.

In a particular embodiment, after installation, the superior bearing surface 506 can be in direct contact with vertebral bone, e.g., cortical bone and cancellous bone. Further, the superior bearing surface 506 can be coated with a bone-growth promoting substance, e.g., a hydroxyapatite coating formed of calcium phosphate. Additionally, the superior bearing surface 506 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 4 through FIG. 9, a projection 508 extends from the superior articular surface 504 of the superior support plate 502. In a particular embodiment, the projection 508 has a hemi-spherical shape. Alternatively, the projection 508 can have an elliptical shape, a cylindrical shape, or other arcuate shape. Moreover, the projection 508 can be formed with a groove 510.

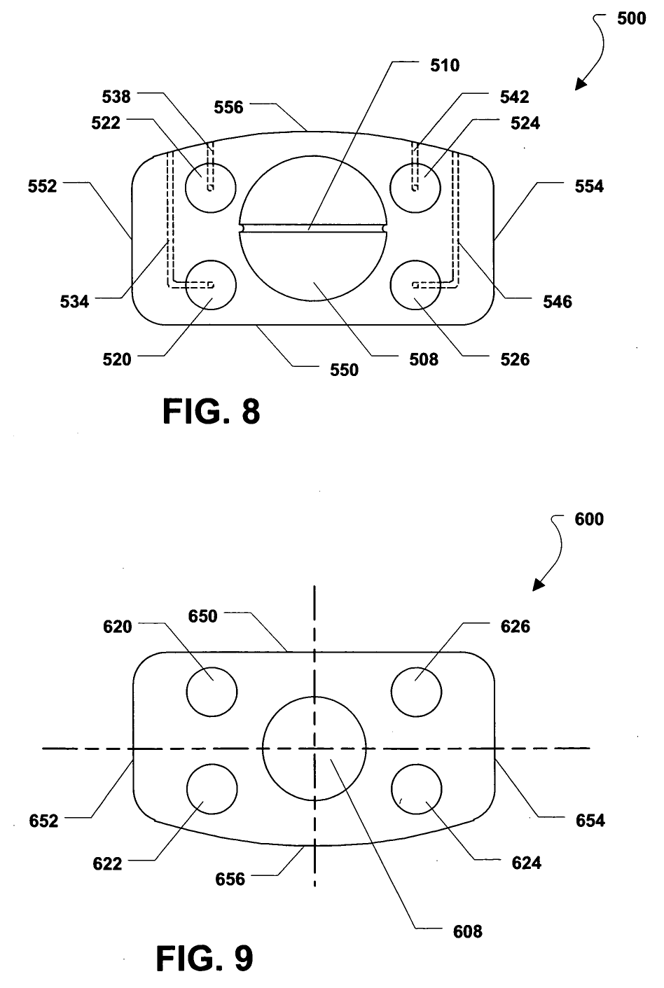

As further illustrated in FIG. 8, the superior component 500 includes a first expandable motion limiter 520, a second expandable motion limiter 522, a third expandable motion limiter 524, and a fourth expandable motion limiter 526 that are affixed, or otherwise attached to, the superior articular surface 504. In a particular embodiment, as depicted in FIG. 8, the expandable motion limiters 520, 522, 524, 526 can be arranged radially around the projection 508. For example, at least two of the expandable motion limiters 520, 522, 524, 526 can be located between a center of the projection 508 and an anterior side of the superior component 500. At least two of the expandable motion limiters 520, 522, 524, 526 can be located between the center of the projection 508 and a posterior side of the superior component 500. Further, at least two of the expandable motion limiters 520, 522, 524, 526 can be located between the center of the projection 508 and a first lateral side. Also, at least two of the expandable motion limiters 520, 522, 524, 526 can be located between the center of the projection 508 and a second lateral side.

FIG. 4 through FIG. 7 indicate that each of the expandable motion limiters 520, 522, 524, 526 can be inflated from a deflated position 528 to one of a plurality of intermediate inflated positions up to a maximum inflated position 530. The expandable motion limiters 520, 522, 524, 526 can be inflated to different positions, the same positions, or a combination thereof. In a particular embodiment, the expandable motion limiters 520, 522, 524, 526 can be inflated with one or more injectable extended use approved medical materials that remain elastic after curing. Further, the injectable extended use approved medical materials can include polymer materials that remain elastic after curing

For example, the polymer materials can include polyurethane materials, polyolefin materials, polyether materials, silicone materials; or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, sterile air, or a combination thereof. In alternative embodiments, the expandable motion limiters can be inflated with one or more of the following: fibroblasts, lipoblasts, chondroblasts, differentiated stem cells, a combination thereof, or another biologic factor which would create a motion limiting tissue when injected into a bioresorbable motion limiting scaffold.

As shown in FIG. 4 through FIG. 8, the superior support plate 502 can include a first port 532 that is in fluid communication with a first fluid channel 534 that provides fluid communication to the first expandable motion limiter 520. The first expandable motion limiter 520 can be inflated with an injectable extended use approved medical material that is delivered to the first expandable motion limiter 520 via the first port 532 and the first fluid channel 534.

As shown, the superior support plate 502 can also include a second port 536 that is in fluid communication with a second fluid channel 536 that provides fluid communication to the second expandable motion limiter 522. The second expandable motion limiter 522 can be inflated with an injectable extended use approved medical material that is delivered to the second expandable motion limiter 522 via the second port 536 and the second fluid channel 536.

FIG. 4 through FIG. 8 also indicate that the superior support plate 502 can include a third port 540 that is in fluid communication with a third fluid channel 542 that provides fluid communication to the third expandable motion limiter 524. The third expandable motion limiter 524 can be inflated with an injectable extended use approved medical material that is delivered to the third expandable motion limiter 524 via the third port 540 and the third fluid channel 542.

The superior support plate 502 can also include a fourth port 544 that is in fluid communication with a fourth fluid channel 546 that provides fluid communication to the fourth expandable motion limiter 526. The fourth expandable motion limiter 526 can be inflated with an injectable extended use approved medical material that is delivered to the fourth expandable motion limiter 526 via the fourth port 544 and the fourth fluid channel 546.

FIG. 4 through FIG. 7 indicate that the superior component 500 can include a superior keel 548 that extends from superior bearing surface 506. During installation, described below, the superior keel 548 can at least partially engage a keel groove that can be established within a cortical rim of a vertebra.

As illustrated in FIG. 8, the superior component 500 can be generally rectangular in shape. For example, the superior component 500 can have a substantially straight posterior side 550. A first straight lateral side 552 and a second substantially straight lateral side 554 can extend substantially perpendicular from the posterior side 550 to an anterior side 556. In a particular embodiment, the anterior side 556 can curve outward such that the superior component 500 is wider through the middle than along the lateral sides 552, 554. Further, in a particular embodiment, the lateral sides 552, 554 are substantially the same length.

FIG. 4 and FIG. 5 show that the superior component 500 includes a first implant inserter engagement hole 560 and a second implant inserter engagement hole 562. In a particular embodiment, the implant inserter engagement holes 560, 562 are configured to receive respective dowels, or pins, that extend from an implant inserter (not shown) that can be used to facilitate the proper installation of an intervertebral prosthetic disc, e.g., the intervertebral prosthetic disc 400 shown in FIG. 4 through FIG. 9.

In a particular embodiment, the inferior component 600 includes an inferior support plate 602 that has an inferior articular surface 604 and an inferior bearing surface 606. In a particular embodiment, the inferior articular surface 604 can be generally curved and the inferior bearing surface 606 can be substantially flat. In an alternative embodiment, the inferior articular surface 604 can be substantially flat and at least a portion of the inferior bearing surface 606 can be generally curved.

In a particular embodiment, after installation, the inferior bearing surface 606 can be in direct contact with vertebral bone, e.g., cortical bone and cancellous bone. Further, the inferior bearing surface 606 can be coated with a bone-growth promoting substance, e.g., a hydroxyapatite coating formed of calcium phosphate. Additionally, the inferior bearing surface 606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 4 through FIG. 7, a depression 608 extends into the inferior articular surface 604 of the inferior support plate 602. In a particular embodiment, the depression 608 is sized and shaped to receive the projection 508 of the superior component 500. For example, the depression 608 can have a hemi-spherical shape. Alternatively, the depression 608 can have an elliptical shape, a cylindrical shape, or other arcuate shape.

The inferior support plate 602 can also include a first motion limiter engagement recess 622, a second motion limiter engagement recess 624, a third motion limiter engagement recess 626, and a fourth motion limiter engagement recess 628. In a particular embodiment, the motion limiter engagement recesses 620, 622, 624, 626 are arranged radially around the depression 608, e.g., in a pattern that mirrors the pattern of the expandable motion limiters 520, 522, 524, 526. Further, each motion limiter engagement recess 620, 622, 624, 626 is sized and shaped to at least partially receive a corresponding expandable motion limiter 520, 522, 524, 526.

In a particular embodiment, each expandable motion limiter 520, 522, 524, 526 cooperates with a respective motion limiter engagement recess 620, 622, 624, 626 in order to limit the motion of the superior component 500 with respect to the inferior component 600. For example, by inflating two expandable motion limiters on one side of the projection 508, a surgeon is able to limit flexion on that side of the projection 508 and as such, limit the relative motion of the superior component 500 with respect to the inferior component 600. Further, this allows the surgeon to limit the motion of a superior vertebra with respect to an inferior vertebra.

The flexibility to alter the range of motion of the intervertebral prosthetic device 400 provided by the expandable motion limiters 520, 522, 524, 526 can allow a surgeon to compensate for a deformity in the segment of the spinal column that includes, or is adjacent to, the superior vertebra and inferior vertebra in question. For example, if a patient's spine is curved in a particular direction, one or more motion limiters 520, 522, 524, 526 opposite the curvature can be inflated to compensate for the curvature. Before or during the surgery, the surgeon can determine any spinal deformity using an X-Ray device, a fluoroscopy device, a computed tomography (CT) device, or any other similar device well known in the art.

FIG. 4 through FIG. 7 indicate that the inferior component 600 can include an inferior keel 648 that extends from inferior bearing surface 606. During installation, described below, the inferior keel 648 can at least partially engage a keel groove that can be established within a cortical rim of a vertebra, e.g., the keel groove 350 shown in FIG. 3.

In a particular embodiment, as shown in FIG. 9, the inferior component 600 can be shaped to match the shape of the superior component 500, shown in FIG. 8. Further, the inferior component 600 can be generally rectangular in shape. For example, the inferior component 600 can have a substantially straight posterior side 650. A first straight lateral side 652 and a second substantially straight lateral side 654 can extend substantially perpendicular from the posterior side 650 to an anterior side 656. In a particular embodiment, the anterior side 656 can curve outward such that the inferior component 600 is wider through the middle than along the lateral sides 652, 654. Further, in a particular embodiment, the lateral sides 652, 654 are substantially the same length.

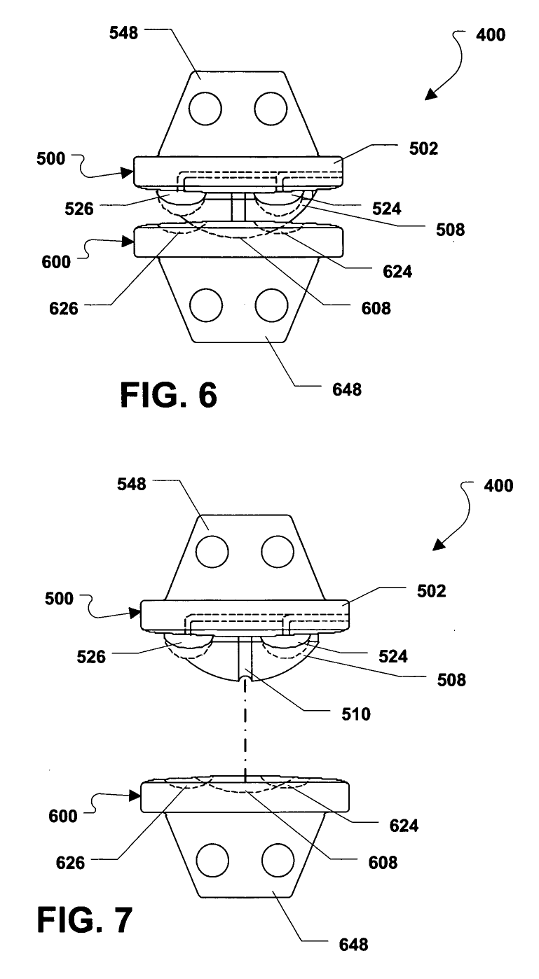

FIG. 4 and FIG. 6 show that the inferior component 600 includes a first implant inserter engagement hole 660 and a second implant inserter engagement hole 662. In a particular embodiment, the implant inserter engagement holes 660, 662 are configured to receive respective dowels, or pins, that extend from an implant inserter (not shown) that can be used to facilitate the proper installation of an intervertebral prosthetic disc, e.g., the intervertebral prosthetic disc 400 shown in FIG. 4 through FIG. 9.

In a particular embodiment, the overall height of the intervertebral prosthetic device 400 can be in a range from fourteen millimeters to forty-six millimeters (14-46 mm). Further, the installed height of the intervertebral prosthetic device 400 can be in a range from eight millimeters to sixteen millimeters (8-16 mm). In a particular embodiment, the installed height can be substantially equivalent to the distance between an inferior vertebra and a superior vertebra when the intervertebral prosthetic device 400 is installed there between.

In a particular embodiment, the length of the intervertebral prosthetic device 400, e.g., along a longitudinal axis, can be in a range from thirty millimeters to forty millimeters (30-40 mm). Additionally, the width of the intervertebral prosthetic device 400, e.g., along a lateral axis, can be in a range from twenty-five millimeters to forty millimeters (25-40 mm). Moreover, in a particular embodiment, each keel 548, 648 can have a height in a range from three millimeters to fifteen millimeters (3-15 mm).

Although depicted in the Figures as a two piece-design, in alternative embodiments, multiple-piece designs can be employed. For example, in an alternative embodiment, the projection 508 is not fixed or unitary with either of the support plates 502, 602 and, instead, is configured as a substantially rigid spherical member (not shown) that can independently articulate with each support plate 502, 602. Additionally or alternatively, each component can comprise multiple components (not shown). These components can articulate with or be fixed to the support plates 502, 602. Furthermore, expandable motion limiters can be configured to limit relative motion between any of the components described above or among multiple components.

Installation of the First Embodiment within an Intervertebral Space

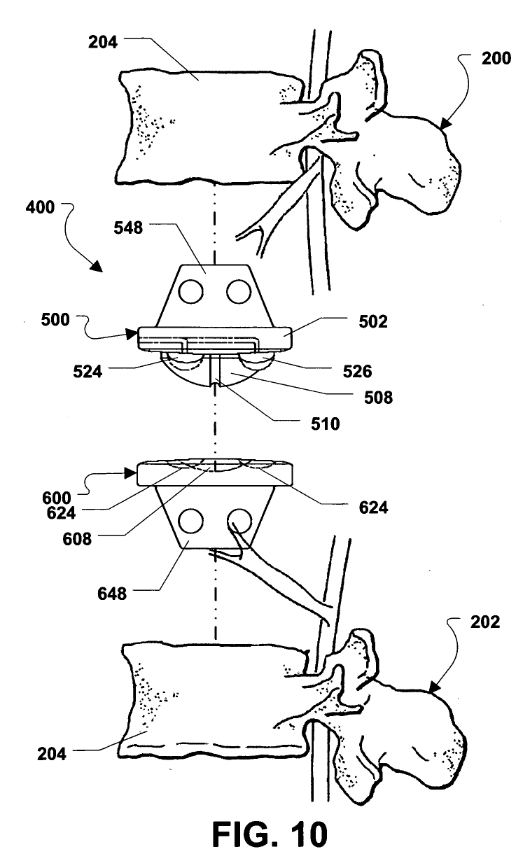

Referring to FIG. 10 through FIG. 12, an intervertebral prosthetic disc is shown between the superior vertebra 200 and the inferior vertebra 202, previously introduced and described in conjunction with FIG. 2. In a particular embodiment, the intervertebral prosthetic disc is the intervertebral prosthetic disc 400 described in conjunction with FIG. 4 through FIG. 9. Alternatively, the intervertebral prosthetic disc can be an intervertebral prosthetic disc according to any of the embodiments disclosed herein.

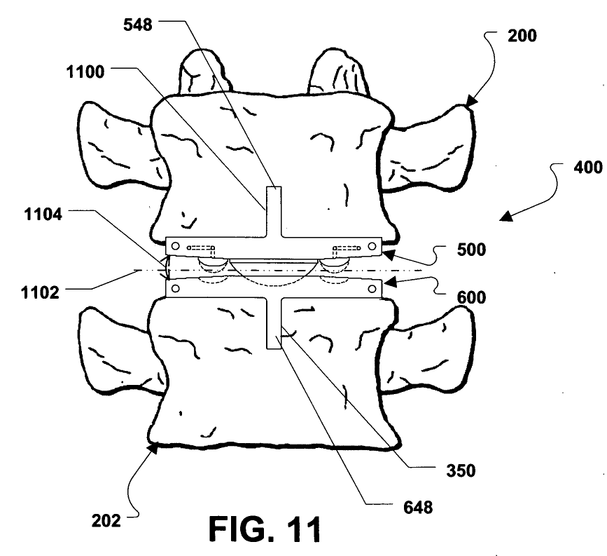

As shown in FIG. 10 through FIG. 12, the intervertebral prosthetic disc 400 is installed within the intervertebral space 214 that can be established between the superior vertebra 200 and the inferior vertebra 202 by removing vertebral disc material (not shown). FIG. 10 shows that the superior keel 548 of the superior component 500 can at least partially engage the cancellous bone and cortical rim of the superior vertebra 200. Further, as shown in FIG. 11, the superior keel 548 of the superior component 500 can at least partially engage a superior keel groove 1100 that can be established within the vertebral body 204 of the superior vertebra 202. In a particular embodiment, the vertebral body 204 can be further cut to allow the superior support plate 502 of the superior component 500 to be at least partially recessed into the vertebral body 204 of the superior vertebra 200.

Also, as shown in FIG. 10, the inferior keel 648 of the inferior component 600 can at least partially engage the cancellous bone and cortical rim of the inferior vertebra 202. Further, as shown in FIG. 11, the inferior keel 648 of the inferior component 600 can at least partially engage the inferior keel groove 350 that can be established within the vertebral body 204 of the inferior vertebra 202. In a particular embodiment, the vertebral body 204 can be further cut to allow the inferior support plate 602 of the inferior component 600 to be at least partially recessed into the vertebral body 204 of the inferior vertebra 200.

As illustrated in FIG. 10 through FIG. 12, the projection 508 that extends from the superior component 500 of the intervertebral prosthetic disc 400 can at least partially engage the depression 608 that is formed within the inferior component 600 of the intervertebral prosthetic disc 400. It is to be appreciated that when the intervertebral prosthetic disc 400 is installed between the superior vertebra 200 and the inferior vertebra 202, the intervertebral prosthetic disc 400 allows relative motion between the superior vertebra 200 and the inferior vertebra 202. Specifically, the configuration of the superior component 500 and the inferior component 600 allows the superior component 500 to rotate with respect to the inferior component 600. As such, the superior vertebra 200 can rotate with respect to the inferior vertebra 202.

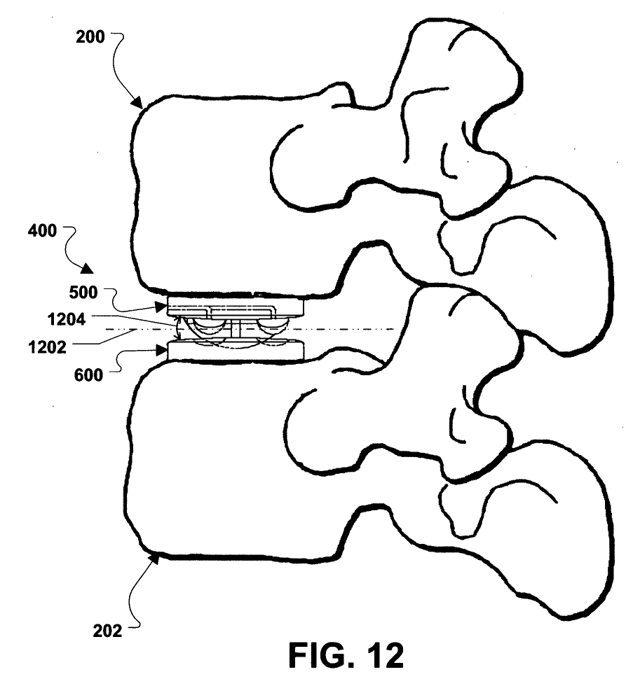

In a particular embodiment, the intervertebral prosthetic disc 400 can allow angular movement in any radial direction relative to the intervertebral prosthetic disc 400. For example, FIG. 11 indicates that the superior component 500 and the inferior component 600 can move relative to each other through a longitudinal axis 1102 over an angle 1104. Additionally, FIG. 12 indicates that the superior component 500 and the inferior component 600 can move relative to each other through a lateral axis 1202 over an angle 1204.

Further, as depicted in FIG. 10 through 12, the inferior component 600 can be placed on the inferior vertebra 202 so that the center of rotation of the inferior component 600 is substantially aligned with the center of rotation of the inferior vertebra 202. Similarly, the superior component 500 can be placed relative to the superior vertebra 200 so that the center of rotation of the superior component 500 is substantially aligned with the center of rotation of the superior vertebra 200. Accordingly, when the vertebral disc, between the inferior vertebra 202 and the superior vertebra 200, is removed and replaced with the intervertebral prosthetic disc 400 the relative motion of the vertebrae 200, 202 provided by the vertebral disc is substantially replicated.

In a particular embodiment, each expandable motion limiter 520, 522, 524, 526 can cooperate with a respective motion limiter engagement recess 620, 622, 624, 626 in order to limit the motion of the superior component 500 with respect to the inferior component 600. However, each expandable motion limiter 520, 522, 524, 526 can be inflated to further limit the relative motion between the superior component 500 and the inferior component 600.

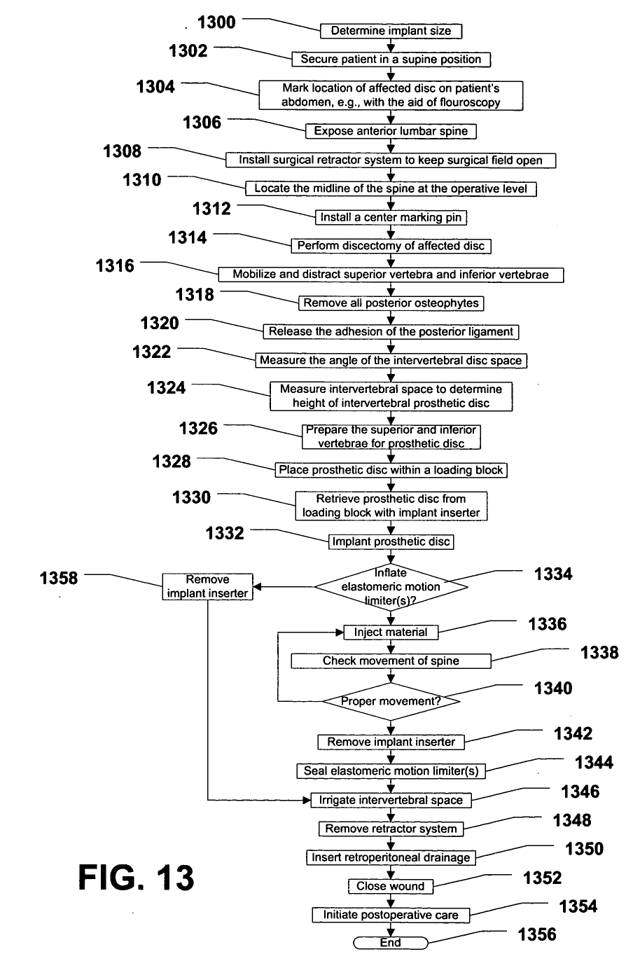

FIG. 13 depicts an exemplary method of installing an intervertebral prosthetic disc between a superior vertebra and an inferior vertebra. Commencing at block 1300, an implant size is determined. For example, the size of the footprint of an intervertebral prosthetic disc to be implanted into a patient can be determined. In a particular embodiment, the implant size can be determined pre-operatively by using computed tomography (CT) and magnetic resonance imaging (MRI) templates. At block 1302, the patient is secured in a supine position to allow an anterior approach to be used to access the patient's spinal column. Further, the patient may be placed in a “French” position in which the patient's legs are spread apart. The “French” position can allow the surgeon to stand between the patient's legs. Further, the “French” position can facilitate proper alignment of the surgical instruments with the patient's spine. In another particular embodiment, the patient can be secured in the supine position on an adjustable surgical table.

In one or more alternative embodiments, a surgeon can use a posterior approach or a lateral approach to implant an intervertebral prosthetic device. As such, the patient may be secured in a different position, e.g., in a prone position for a posterior approach or in a lateral decubitus position for a lateral approach.

Moving to block 1304, the location of the affected disc is marked on patient's abdomen, e.g., with the aid of fluoroscopy. At block 1306, the patient's anterior lumbar spine is exposed. The anterior lumbar spine can be approached through a transperitoneal or a retroperitoneal exposure using the appropriate instruments and retractors. For example, an anterior approach can be facilitated with the aid of a surgical retractor system, e.g., the Medtronic Sofamor Danek Endoring™ Surgical Retractor System. At block 1308, a surgical retractor system can be installed to keep the surgical field open during the surgery.

Proceeding to block 1310, the midline of the spine at the operative level is located. For example, the midline of the spine can be located using an intra-operative anterior-posterior (A-P) image. At block 1312, once the midline is located, a center marking pin can be installed. Moving to block 1314, a discectomy of the affected disc can be performed. At block 1316, the superior vertebra and inferior vertebra can be mobilized and distracted. Further, at block 1318, all posterior osteophytes can be removed.

Moving to block 1320, the adhesion of the posterior ligament can be released from the superior vertebra and inferior vertebra. At block 1322, the angle of the intervertebral disc space is measured. Moreover, at block 1324, the intervertebral space is measured to determine a height of an intervertebral prosthetic disc to be implanted into the patient, e.g., into the intervertebral space between the superior vertebra and the inferior vertebra. At block 1326, the superior and inferior vertebrae are prepared to receive a prosthetic disc, e.g., an intervertebral prosthetic disc according to one or more of the embodiments described herein. In a particular embodiment, the preparation of the superior and inferior vertebrae may include removing portions of the cortical rim of each vertebra. Further, the preparation may include cutting one or more keel grooves in the cortical rim of each vertebra.

Proceeding to block 1328, the prosthetic disc can be placed within a loading block. At block 1330, the prosthetic disc can be retrieved from the loading block using an implant inserter that is designed to engage a prosthetic disc, e.g., an intervertebral prosthetic disc according to one or more of the embodiments described herein. Moving to block 1332, a prosthetic disc can be implanted.

At decision step 1334, it can be determined whether to inflate one or more of the expandable motion limiters that are incorporated into the design of the intervertebral prosthetic disc. In a particular embodiment, that determination can be at least partially based on one or more X-rays taken prior to the surgery or during the surgery. Additionally, that determination can be at least partially based on an inspection of the patient's spine during the surgery. Further, that determination can be at least partially based on one or more measurements taken during the surgery.

If it is determined to inflate one or more of the expandable motion limiters, the method proceeds to block 1336 and an injectable extended use approved medical material can be injected into one or more of the expandable motion limiters. Accordingly, the one or more expandable motion limiters can be inflated from a deflated position to one of a plurality of inflated positions—up to a maximum inflated position. In a particular embodiment, the volume of material that is injected into the one or more expandable motion limiters can be used to determine the inflated position of the one or more expandable motion limiters. Alternatively, the pressure of the material that is injected into the one or more expandable motion limiters can be used to determine the inflated position of the one or more expandable motion limiters.

At block 1338, the movement of the patient's spine is checked. For example, the adjustable surgical table can be moved in order to slightly flex the patient's spine. Moving to decision step 1340, it can be determined whether the movement is proper, i.e., whether the expandable motion limiters are properly limiting the motion of the patient's spine. Further, it can be determined whether the expandable motion limiters are properly limiting the motion of the superior vertebra with respect to the inferior vertebra.

At decision step 1340, if the one or more expandable motion limiters are not properly limiting the motion of the patient's spine, the method can return to block 1336 and more injectable extended use approved medical material can be injected into the one or more expandable motion limiters. As such, each of the one or more expandable motion limiters can be inflated from a first inflated position to a second inflated position. From block 1336, the method can continue as described herein. On the other hand, at decision step 1340, if the one or more expandable motion limiters are properly limiting the motion of the patient's spine, the method continues to block 1342 and the implant inserter can be removed from the intervertebral prosthetic disc and the surgical field.

Moving to block 1344, the one or more expandable motion limiters can be sealed. In one embodiment, a screw can be inserted into each port associated with each expandable motion limiter. In another embodiment, the polymer may be self-sealing, i.e., a polymer may be used that can cure under the ambient conditions of the surgery. In such an embodiment, the polymer can cure within each fluid channel through which the polymer can be injected and block the fluid channel. In yet another embodiment, a one-way valve can be installed within each fluid channel of the intervertebral prosthetic disc adjacent to, or downstream from, each port. As such, each one-way valve can allow polymer to be injected into the intervertebral prosthetic disc and prevent the polymer from be extruded from the intervertebral prosthetic disc.

Continuing to block 1346, the intervertebral space can be irrigated. Further, at block 1348, the retractor system can be removed. At block 1350, a retroperitoneal drainage can be inserted into the wound. Additionally, at block 1352, the wound can be closed. Moving to block 1354, postoperative care can be initiated. The method ends at step 1556.

Returning to decision step 1334, when it is determined not to inflate one or more of the expandable motion limiters, the method proceeds to block 1358 and the implant inserter is removed. The method can move to block 1346 and continue as described herein.

### Description of a Second Embodiment of an Intervertebral Prosthetic Disc

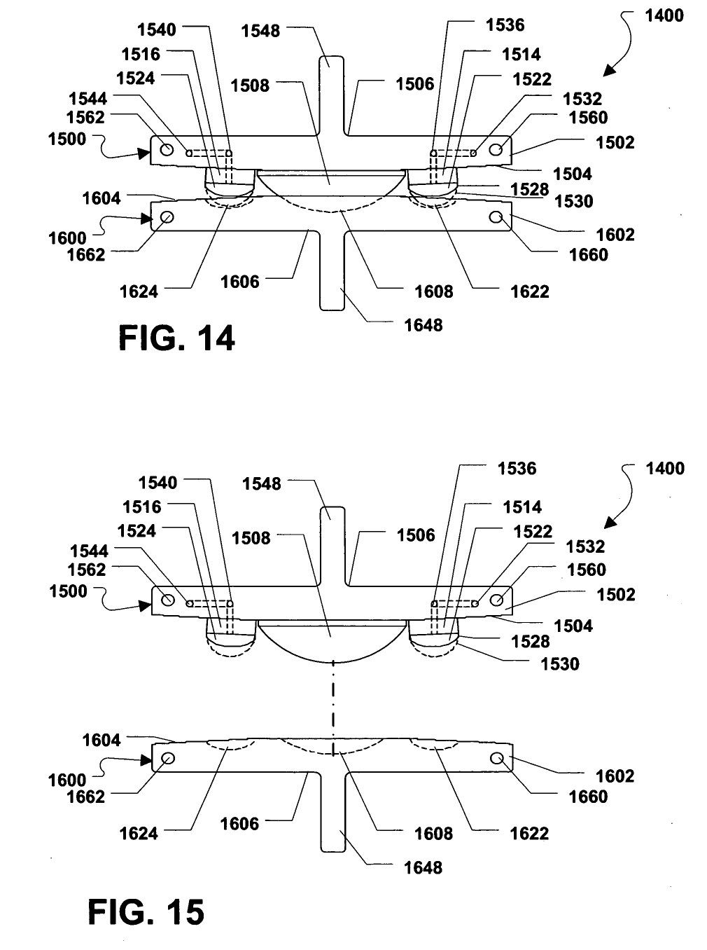

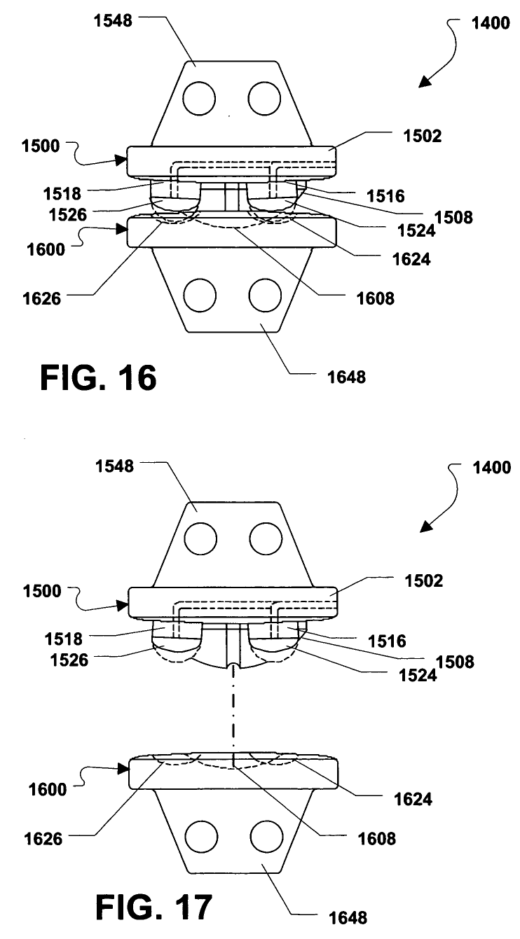

Referring to FIGS. 14 through 19 a second embodiment of an intervertebral prosthetic disc is shown and is generally designated 1400. As illustrated, the intervertebral prosthetic disc 1400 includes a superior component 1500 and an inferior component 1600. In a particular embodiment, the components 1500, 1600 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 1500, 1600 can be made from any other substantially rigid biocompatible materials.

In a particular embodiment, the superior component 1500 includes a superior support plate 1502 that has a superior articular surface 1504 and a superior bearing surface 1506. In a particular embodiment, the superior articular surface 1504 can be generally curved and the superior bearing surface 1506 can be substantially flat. In an alternative embodiment, the superior articular surface 1504 can be substantially flat and at least a portion of the superior bearing surface 1506 can be generally curved.

In a particular embodiment, after installation, the superior bearing surface 1506 can be in direct contact with vertebral bone, e.g., cortical bone and cancellous bone. Further, the superior bearing surface 1506 can be coated with a bone-growth promoting substance, e.g., a hydroxyapatite coating formed of calcium phosphate. Additionally, the superior bearing surface 1506 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 14 through FIG. 19, a projection 1508 extends from the superior articular surface 1504 of the superior support plate 1502. In a particular embodiment, the projection 1508 has a hemi-spherical shape. Alternatively, the projection 1508 can have an elliptical shape, a cylindrical shape, or other arcuate shape. Additionally, the projection 1508 can be formed with a groove 1510.

FIG. 14 through FIG. 19 show that the superior component 1500 can include a first motion limiting post 1512, a second motion limiting post 1514, a third motion limiting post 1516, and a fourth motion limiting post 1518 that extend from the superior articular surface 1504. In a particular embodiment, the motion limiting posts 1512 are disposed radially around the projection 1508. For example, at least two of the motion limiting posts 1512, 1514, 1516, 1518 can be located between a center of the projection 1508 and an anterior side of the superior component 1500. At least two of the motion limiting posts 1512, 1514, 1516, 1518 can be located between the center of the projection 1508 and a posterior side of the superior component 1500. Further, at least two of the motion limiting posts 1512, 1514, 1516, 1518 can be located between the center of the projection 1508 and a first lateral side. Also, at least two of the motion limiting posts 1512, 1514, 1516, 1518 can be located between the center of the projection 1508 and a second lateral side.

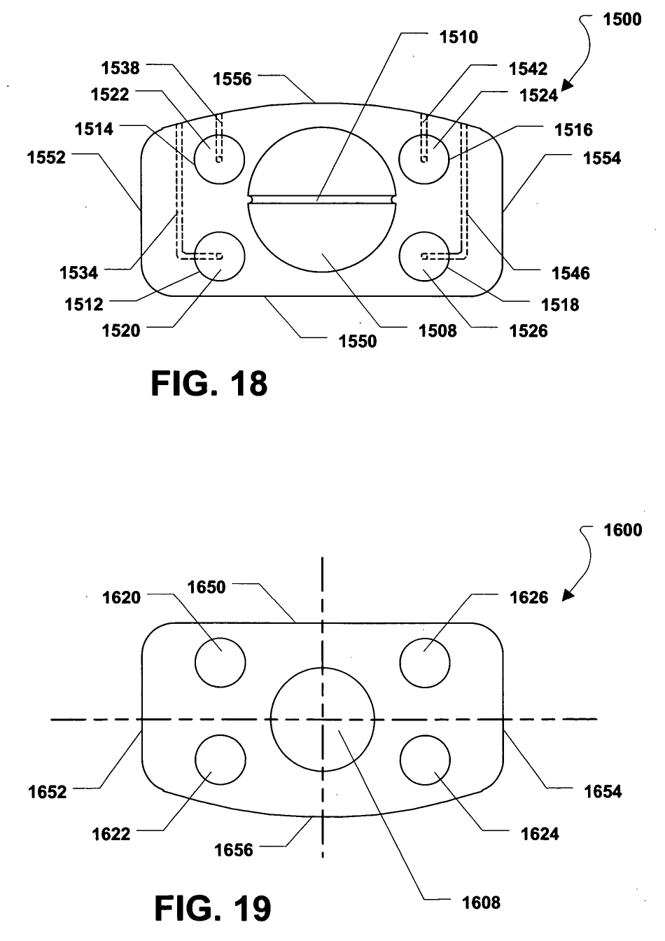

As further illustrated in FIG. 18, the superior component 1500 includes a first expandable motion limiter 1520 that can be affixed, or otherwise attached, to the first motion limiting post 1512. A second expandable motion limiter 1522 can be affixed, or otherwise attached, to the second motion limiting post 1514. A third expandable motion limiter 1524 can be affixed, or otherwise attached, to the third motion limiting post 1516. Additionally, a fourth expandable motion limiter 1526 can be affixed, or otherwise attached, to the fourth motion limiting post 1518.

FIG. 14 through FIG. 19 indicate that each of the expandable motion limiters 1520, 1522, 1524, 1526 can be inflated from a deflated position 1528 to one of a plurality of intermediate inflated positions up to a maximum inflated position 1530. The expandable motion limiters 1520, 1522, 1524, 1526 can be inflated to different positions, the same positions, or a combination thereof. In a particular embodiment, the expandable motion limiters 1520, 1522, 1524, 1526 can be inflated with an injectable extended use approved medical materials that remain elastic after curing. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, sterile air, or a combination thereof. In further alternative embodiments, the expandable motion limiters can be inflated with one or more of the following: fibroblasts, lipoblasts, chondroblasts, differentiated stem cells, a combination thereof, or another biologic factor which would create a motion limiting tissue when injected into a bioresorbable motion limiting scaffold.

As shown in FIG. 14 through FIG. 18, the superior support plate 1502 can include a first port 1532 that is in fluid communication with a first fluid channel 1534 that provides fluid communication to the first expandable motion limiter 1520. The first expandable motion limiter 1520 can be inflated with an injectable extended use approved medical material that is delivered to the first expandable motion limiter 1520 via the first port 1532 and the first fluid channel 1534.

As shown, the superior support plate 1502 can also include a second port 1536 that is in fluid communication with a second fluid channel 1536 that provides fluid communication to the second expandable motion limiter 1522. The second expandable motion limiter 1522 can be inflated with an injectable extended use approved medical material that is delivered to the second expandable motion limiter 1522 via the second port 1536 and the second fluid channel 1536.

FIG. 14 through FIG. 18 also indicate that the superior support plate 1502 can include a third port 1540 that is in fluid communication with a third fluid channel 1542 that provides fluid communication to the third expandable motion limiter 1524. The third expandable motion limiter 1524 can be inflated with an injectable extended use approved medical material that is delivered to the third expandable motion limiter 1524 via the third port 1540 and the third fluid channel 1542.

The superior support plate 1502 can also include a fourth port 1544 that is in fluid communication with a fourth fluid channel 1546 that provides fluid communication to the fourth expandable motion limiter 1526. The fourth expandable motion limiter 1526 can be inflated with an injectable extended use approved medical material that is delivered to the fourth expandable motion limiter 1526 via the fourth port 1544 and the fourth fluid channel 1546.

FIG. 14 through FIG. 17 indicate that the superior component 1500 can include a keel 1548 that extends from superior bearing surface 1506. During installation, described below, the keel 1548 can at least partially engage a keel groove that can be established within a cortical rim of a vertebra.

As illustrated in FIG. 18, the superior component 1500 can be generally rectangular in shape. For example, the superior component 1500 can have a substantially straight posterior side 1550. A first straight lateral side 1552 and a second substantially straight lateral side 1554 can extend substantially perpendicular from the posterior side 1550 to an anterior side 1556. In a particular embodiment, the anterior side 1556 can curve outward such that the superior component 1500 is wider through the middle than along the lateral sides 1552, 1554. Further, in a particular embodiment, the lateral sides 1552, 1554 are substantially the same length.

As depicted in FIG. 14 through FIG. 19, the inferior component 1600 includes an inferior support plate 1602 that has an inferior articular surface 1604 and an inferior bearing surface 1606. In a particular embodiment, the inferior articular surface 1604 can be generally curved and the inferior bearing surface 1606 can be substantially flat. In an alternative embodiment, the inferior articular surface 1604 can be flat and at least a portion of the inferior bearing surface 1606 can be curved.

In a particular embodiment, after installation, the inferior bearing surface 1606 can be in direct contact with vertebral bone, e.g., cortical bone and cancellous bone. Further, the inferior bearing surface 1606 can be coated with a bone-growth promoting substance, e.g., a hydroxyapatite coating formed of calcium phosphate. Additionally, the inferior bearing surface 1606 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 14 through FIG. 17, a depression 1608 extends into the inferior articular surface 1604 of the inferior support plate 1602. In a particular embodiment, the depression 1608 is sized and shaped to receive the projection 1508 of the inferior component 1600. For example, the depression 1608 can have a hemi-spherical shape. Alternatively, the depression 1608 can have an elliptical shape, a cylindrical shape, or other arcuate shape.

The inferior support plate 1602 can also include a first motion limiter engagement recess 1622, a second motion limiter engagement recess 1624, a third motion limiter engagement recess 1626, and a fourth motion limiter engagement recess 1628. In a particular embodiment, the motion limiter engagement recesses 1620, 1622, 1624, 1626 are arranged radially around the depression 1608, e.g., in a pattern that mirrors the pattern of the expandable motion limiters 1520, 1522, 1524, 1526. Further, each motion limiter engagement recess 1620, 1622, 1624, 1626 is sized and shaped to at least partially receive a corresponding expandable motion limiter 1520, 1522, 1524, 1526.

In a particular embodiment, each expandable motion limiter 1520, 1522, 1524, 1526 cooperates with a respective motion limiter engagement recess 1620, 1622, 1624, 1626 in order to limit the motion of the superior component 1500 with respect to the inferior component 1600. For example, by inflating two expandable motion limiters on one side of the projection 1508, a surgeon is able to limit flexion on that side of the projection 1508 and as such, limit the relative motion of the superior component 1500 with respect to the inferior component 1600. Further, this allows the surgeon to limit the motion of a superior vertebra with respect to an inferior vertebra.

The flexibility to alter the range of motion of the intervertebral prosthetic device 1400 provided by the expandable motion limiters 1520, 1522, 1524, 1526 can allow a surgeon to compensate for a deformity in the segment of the spinal column that includes, or is adjacent to, the superior vertebra and inferior vertebra in question. For example, if a patient's spine is curved in a particular direction, one or more motion limiters 1520, 1522, 1524, 1526 opposite the curvature can be inflated to compensate for the curvature. Before or during the surgery, the surgeon can determine any spinal deformity using an X-Ray device, a fluoroscopy device, a computed tomography (CT) device, or any other similar device well known in the art.

FIG. 14 through FIG. 17 indicate that the inferior component 1600 can include a keel 1648 that extends from inferior bearing surface 1606. After installation, the keel 1648 can at least partially engage a keel groove that can be established within a cortical rim of a vertebra.

In a particular embodiment, as shown in FIG. 19, the inferior component 1600 can be shaped to match the shape of the inferior component 1600, shown in FIG. 18. Further, the inferior component 1600 can be generally rectangular in shape. For example, the inferior component 1600 can have a substantially straight posterior side 1650. A first straight lateral side 1652 and a second substantially straight lateral side 1654 can extend substantially perpendicular from the posterior side 1650 to an anterior side 1656. In a particular embodiment, the anterior side 1656 can curve outward such that the inferior component 1600 is wider through the middle than along the lateral sides 1652, 1654. Further, in a particular embodiment, the lateral sides 1652, 1654 are substantially the same length.

In a particular embodiment, the overall height of the intervertebral prosthetic device 1400 can be in a range from fourteen millimeters to forty-six millimeters (14-46 mm). Further, the installed height of the intervertebral prosthetic device 1400 can be in a range from eight millimeters to sixteen millimeters (8-16 mm). In a particular embodiment, the installed height can be substantially equivalent to the distance between an inferior vertebra and a superior vertebra when the intervertebral prosthetic device 1400 is installed there between.

In a particular embodiment, the length of the intervertebral prosthetic device 1400, e.g., along a longitudinal axis, can be in a range from thirty millimeters to forty millimeters (30-40 mm). Additionally, the width of the intervertebral prosthetic device 400, e.g., along a lateral axis, can be in a range from twenty-five millimeters to forty millimeters (25-40 mm). Moreover, in a particular embodiment, each keel 1548, 1648 can have a height in a range from three millimeters to fifteen millimeters (3-15 mm).

Although depicted in the Figures as a two piece-design, in alternative embodiments, multiple-piece designs can be employed. For example, in an alternative embodiment, the projection 1508 is not fixed or unitary with either of the support plates 1502, 1602 and, instead, is configured as a substantially rigid spherical member (not shown) that can independently articulate with each support plate 1502, 1602. Additionally or alternatively, each component can comprise multiple components (not shown). These components can articulate with or be fixed to the support plates 1502, 1602. Furthermore, expandable motion limiters can be configured to limit relative motion between any of the components described above or among multiple components.

### Description of a Third Embodiment of an Intervertebral Prosthetic Disc

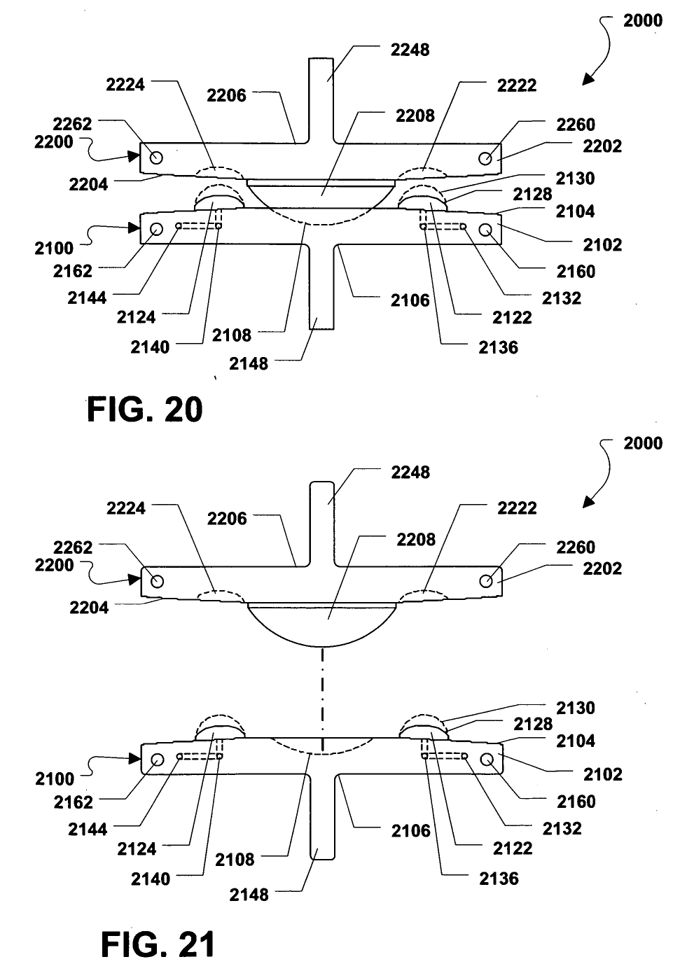

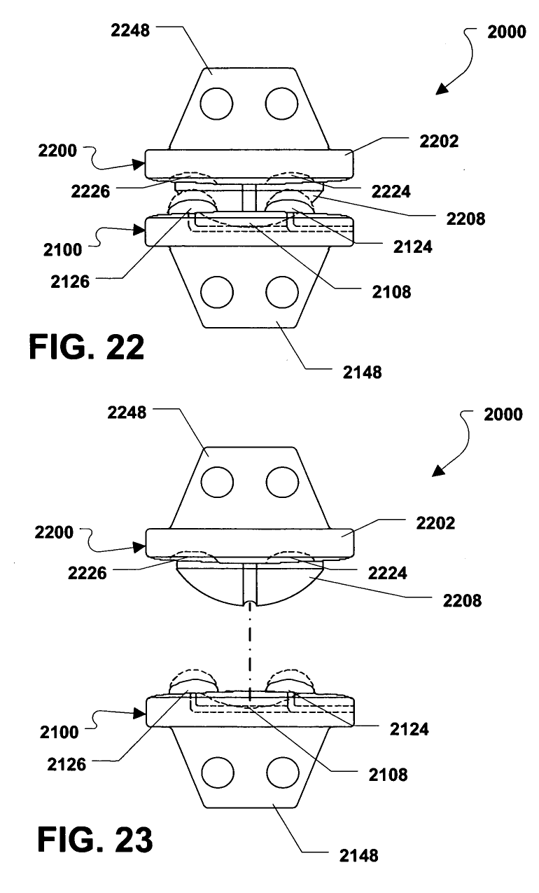

Referring to FIGS. 20 through 25 a third embodiment of an intervertebral prosthetic disc is shown and is generally designated 2000. As illustrated, the intervertebral prosthetic disc 2000 includes an inferior component 2100 and a superior component 2200. In a particular embodiment, the components 2100, 2200 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 2100, 2200 can be made from any other substantially rigid biocompatible materials.

In a particular embodiment, the inferior component 2100 includes an inferior support plate 2102 that has an inferior articular surface 2104 and an inferior bearing surface 2106. In a particular embodiment, the inferior articular surface 2104 can be generally curved and the inferior bearing surface 2106 can be substantially flat. In an alternative embodiment, the inferior articular surface 2104 can be substantially flat and at least a portion of the inferior bearing surface 2106 can be generally curved.

In a particular embodiment, after installation, the inferior bearing surface 2106 can be in direct contact with vertebral bone, e.g., cortical bone and cancellous bone. Further, the inferior bearing surface 2106 can be coated with a bone-growth promoting substance, e.g., a hydroxyapatite coating formed of calcium phosphate. Additionally, the inferior bearing surface 2106 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 20 through FIG. 23, a depression 2108 extends into the inferior articular surface 2104 of the inferior support plate 2102. For example, the depression 2108 can have a hemi-spherical shape. Alternatively, the depression 2108 can have an elliptical shape, a cylindrical shape, or other arcuate shape.

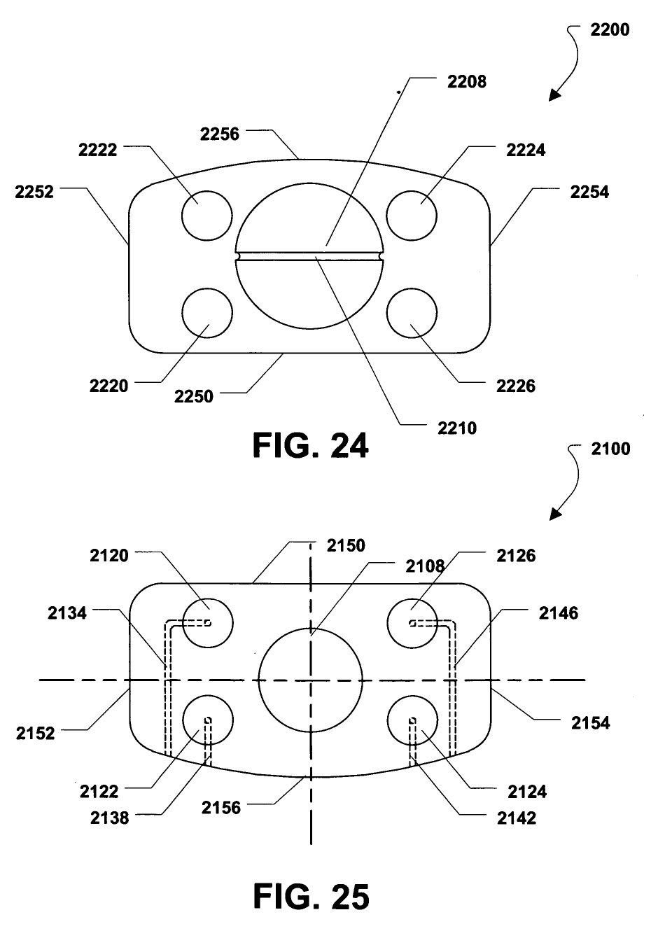

As further illustrated in FIG. 24, the inferior component 2100 includes a first expandable motion limiter 2120, a second expandable motion limiter 2122, a third expandable motion limiter 2124, and a fourth expandable motion limiter 2126 that are affixed, or otherwise attached to, the inferior articular surface 2104. In a particular embodiment, as depicted in FIG. 24, the expandable motion limiters 2120, 2122, 2124, 2126 can be arranged radially around the depression 2108.

FIG. 20 through FIG. 23 indicate that each of the expandable motion limiters 2120, 2122, 2124, 2126 can be inflated from a deflated position 2128 to one of a plurality of intermediate inflated positions up to a maximum inflated position 2130. The expandable motion limiters 2120, 2122, 2124, 2126 can be inflated to different positions, the same positions, or a combination thereof. In a particular embodiment, the expandable motion limiters 2120, 2122, 2124, 2126 can be inflated with an injectable extended use approved medical materials that remain elastic after curing. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, sterile air, or a combination thereof. In alternative embodiments, the expandable motion limiters can be inflated with one or more of the following: fibroblasts, lipoblasts, chondroblasts, differentiated stem cells, a combination thereof, or another biologic factor which would create a motion limiting tissue when injected into a bioresorbable motion limiting scaffold.

As shown in FIG. 20 through FIG. 24, the inferior support plate 2102 can include a first port 2132 that is in fluid communication with a first fluid channel 2134 that provides fluid communication to the first expandable motion limiter 2120. The first expandable motion limiter 2120 can be inflated with an injectable extended use approved medical material that is delivered to the first expandable motion limiter 2120 via the first port 2132 and the first fluid channel 2134.

As shown, the inferior support plate 2102 can also include a second port 2136 that is in fluid communication with a second fluid channel 2136 that provides fluid communication to the second expandable motion limiter 2122. The second expandable motion limiter 2122 can be inflated with an injectable extended use approved medical material that is delivered to the second expandable motion limiter 2122 via the second port 2136 and the second fluid channel 2136.

FIG. 20 through FIG. 24 also indicate that the inferior support plate 2102 can include a third port 2140 that is in fluid communication with a third fluid channel 2142 that provides fluid communication to the third expandable motion limiter 2124. The third expandable motion limiter 2124 can be inflated with an injectable extended use approved medical material that is delivered to the third expandable motion limiter 2124 via the third port 2140 and the third fluid channel 2142.

The inferior support plate 2102 can also include a fourth port 2144 that is in fluid communication with a fourth fluid channel 2146 that provides fluid communication to the fourth expandable motion limiter 2126. The fourth expandable motion limiter 2126 can be inflated with an injectable extended use approved medical material that is delivered to the fourth expandable motion limiter 2126 via the fourth port 2144 and the fourth fluid channel 2146.

FIG. 20 through FIG. 23 indicate that the inferior component 2100 can include an inferior keel 2148 that extends from inferior bearing surface 2106. After installation, the inferior keel 2148 can at least partially engage a keel groove that can be established within a cortical rim of a vertebra.

In a particular embodiment, as shown in FIG. 25, the inferior component 2100 can be generally rectangular in shape. For example, the inferior component 2100 can have a substantially straight posterior side 2150. A first straight lateral side 2152 and a second substantially straight lateral side 2154 can extend substantially perpendicular from the posterior side 2150 to an anterior side 2156. In a particular embodiment, the anterior side 2156 can curve outward such that the inferior component 2100 is wider through the middle than along the lateral sides 2152, 2154. Further, in a particular embodiment, the lateral sides 2152, 2154 are substantially the same length.

FIG. 20 and FIG. 21 show that the inferior component 2100 includes a first implant inserter engagement hole 2160 and a second implant inserter engagement hole 2162. In a particular embodiment, the implant inserter engagement holes 2160, 2162 are configured to receive respective dowels, or pins, that extend from an implant inserter (not shown) that can be used to facilitate the proper installation of an intervertebral prosthetic disc, e.g., the intervertebral prosthetic disc 1600 shown in FIG. 20 through FIG. 25.

In a particular embodiment, the superior component 2200 includes a superior support plate 2202 that has a superior articular surface 2204 and a superior bearing surface 2206. In a particular embodiment, the superior articular surface 2204 can be generally curved and the superior bearing surface 2206 can be substantially flat. In an alternative embodiment, the superior articular surface 2204 can be flat and at least a portion of the superior bearing surface 2206 can be curved.

In a particular embodiment, after installation, the superior bearing surface 2206 can be in direct contact with vertebral bone, e.g., cortical bone and cancellous bone. Further, the superior bearing surface 2206 can be coated with a bone-growth promoting substance, e.g., a hydroxyapatite coating formed of calcium phosphate. Additionally, the superior bearing surface 2206 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 20 through FIG. 25, a projection 2208 extends from the superior articular surface 2204 of the superior support plate 2202. In a particular embodiment, the projection 2208 is sized and shaped to engage the depression 2108 of the inferior component 2100. In a particular embodiment, the projection 2208 has a hemi-spherical shape. Alternatively, the projection 2208 can have an elliptical shape, a cylindrical shape, or other arcuate shape. Also, the projection 2208 can be formed with a groove 2210.

The superior support plate 2202 can also include a first motion limiter engagement recess 2222, a second motion limiter engagement recess 2224, a third motion limiter engagement recess 2226, and a fourth motion limiter engagement recess 2228. In a particular embodiment, the motion limiter engagement recesses 2220, 2222, 2224, 2226 are arranged radially around the depression 2208, e.g., in a pattern that mirrors the pattern of the expandable motion limiters 2120, 2122, 2124, 2126. Further, each motion limiter engagement recess 2220, 2222, 2224, 2226 is sized and shaped to at least partially receive a corresponding expandable motion limiter 2120, 2122, 2124, 2126.

In a particular embodiment, each expandable motion limiter 2120, 2122, 2124, 2126 cooperates with a respective motion limiter engagement recess 2220, 2222, 2224, 2226 in order to limit the motion of the inferior component 2100 with respect to the superior component 2200. For example, by inflating two expandable motion limiters on one side of the depression 2108, a surgeon is able to limit flexion on that side of the depression 2108 and as such, limit the relative motion of the inferior component 2100 with respect to the superior component 2200. Further, this allows the surgeon to limit the motion of a superior vertebra with respect to an inferior vertebra.

The flexibility to alter the range of motion of the intervertebral prosthetic device 2000 provided by the expandable motion limiters 2120, 2122, 2124, 2126 can allow a surgeon to compensate for a deformity in the segment of the spinal column that includes, or is adjacent to, the superior vertebra and inferior vertebra in question. For example, if a patient's spine is curved in a particular direction, one or more motion limiters 2120, 2122, 2124, 2126 opposite the curvature can be inflated to compensate for the curvature. Before or during the surgery, the surgeon can determine any spinal deformity using an X-Ray device, a fluoroscopy device, a computed tomography (CT) device, or any other similar device well known in the art.

FIG. 20 through FIG. 23 indicate that the superior component 2200 can include a keel 2248 that extends from superior bearing surface 2206. After installation, the keel 2248 can at least partially engage a keel groove that can be established within a cortical rim of a vertebra.

As illustrated in FIG. 24, the superior component 2200 can be shaped to match the shape of the inferior component 2100, shown in FIG. 25. Further, the superior component 2200 can be generally rectangular in shape. For example, the superior component 2200 can have a substantially straight posterior side 2250. A first straight lateral side 2252 and a second substantially straight lateral side 2254 can extend substantially perpendicular from the posterior side 2250 to an anterior side 2256. In a particular embodiment, the anterior side 2256 can curve outward such that the superior component 2200 is wider through the middle than along the lateral sides 2252, 2254. Further, in a particular embodiment, the lateral sides 2252, 2254 are substantially the same length.

FIG. 20 and FIG. 21 show that the superior component 2200 includes a first implant inserter engagement hole 2260 and a second implant inserter engagement hole 2262. In a particular embodiment, the implant inserter engagement holes 2260, 2262 are configured to receive respective dowels, or pins, that extend from an implant inserter (not shown) that can be used to facilitate the proper installation of an intervertebral prosthetic disc, e.g., the intervertebral prosthetic disc 2000 shown in FIG. 20 through FIG. 25.

In a particular embodiment, the overall height of the intervertebral prosthetic device 2000 can be in a range from fourteen millimeters to forty-six millimeters (14-46 mm). Further, the installed height of the intervertebral prosthetic device 2000 can be in a range from eight millimeters to sixteen millimeters (8-16 mm). In a particular embodiment, the installed height can be substantially equivalent to the distance between an inferior vertebra and a superior vertebra when the intervertebral prosthetic device 2000 is installed there between.

In a particular embodiment, the length of the intervertebral prosthetic device 2000, e.g., along a longitudinal axis, can be in a range from thirty millimeters to forty millimeters (30-40 mm). Additionally, the width of the intervertebral prosthetic device 2000, e.g., along a lateral axis, can be in a range from twenty-five millimeters to forty millimeters (25-40 mm). Moreover, in a particular embodiment, each keel 2148, 2248 can have a height in a range from three millimeters to fifteen millimeters (3-15 mm).

Although depicted in the Figures as a two piece-design, in alternative embodiments, multiple-piece designs can be employed. For example, in an alternative embodiment, the projection 2208 is not fixed or unitary with either of the support plates 2102, 2202 and, instead, is configured as a substantially rigid spherical member (not shown) that can independently articulate with each support plate 2102, 2202. Additionally or alternatively, each component can comprise multiple components (not shown). These components can articulate with or be fixed to the support plates 2102, 2202. Furthermore, expandable motion limiters can be configured to limit relative motion between any of the components described above or among multiple components.

### Description of a Fourth Embodiment of an Intervertebral Prosthetic Disc

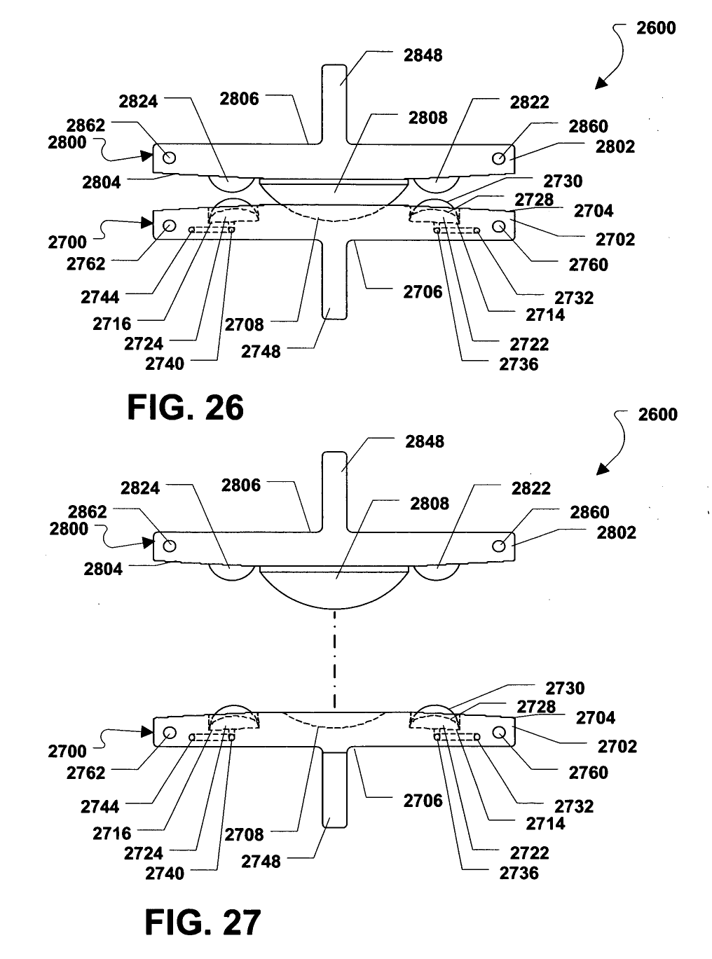

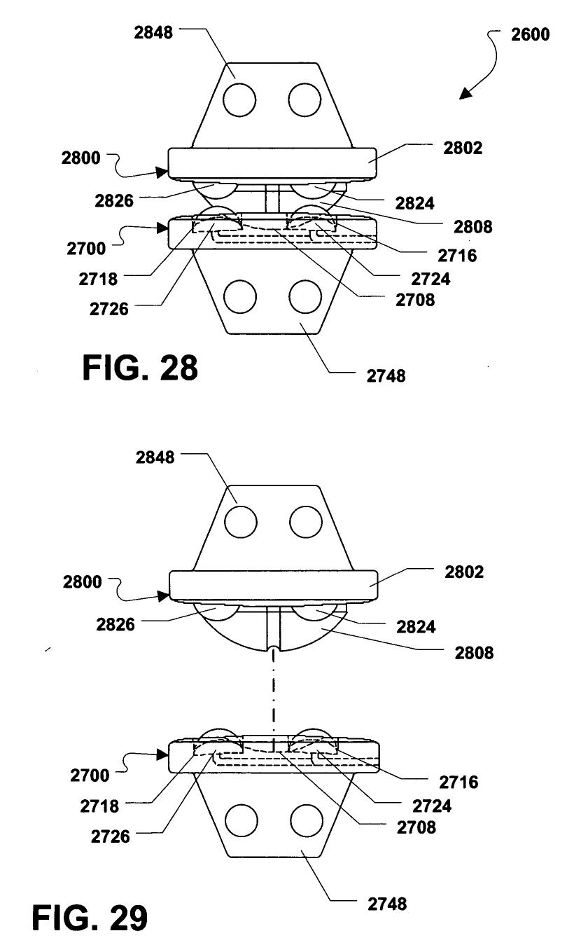

Referring to FIGS. 26 through 31 a fourth embodiment of an intervertebral prosthetic disc is shown and is generally designated 2600. As illustrated, the intervertebral prosthetic disc 2600 includes an inferior component 2700 and a superior component 2800. In a particular embodiment, the components 2700, 2800 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 2700, 2800 can be made from any other substantially rigid biocompatible materials.

In a particular embodiment, the inferior component 2700 includes an inferior support plate 2702 that has an inferior articular surface 2704 and an inferior bearing surface 2706. In a particular embodiment, the inferior articular surface 2704 can be generally curved and the inferior bearing surface 2706 can be substantially flat. In an alternative embodiment, the inferior articular surface 2704 can be substantially flat and at least a portion of the inferior bearing surface 2706 can be generally curved.

In a particular embodiment, after installation, the inferior bearing surface 2706 can be in direct contact with vertebral bone, e.g., cortical bone and cancellous bone. Further, the inferior bearing surface 2706 can be coated with a bone-growth promoting substance, e.g., a hydroxyapatite coating formed of calcium phosphate. Additionally, the inferior bearing surface 2706 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 26 through FIG. 23, a depression 2708 extends into the inferior articular surface 2704 of the inferior support plate 2702. For example, the depression 2708 can have a hemi-spherical shape. Alternatively, the depression 2708 can have an elliptical shape, a cylindrical shape, or other arcuate shape.

FIG. 26 through FIG. 31 show that the inferior component 2700 can include a first motion limiter recess 2712, a second motion limiter recess 2714, a third motion limiter recess 2716, and a fourth motion limiter recess 2718 that can be formed within the inferior articular surface 2704. In a particular embodiment, the motion limiter recesses 2712 are disposed radially around the depression 2708. For example, at least two of the motion limiter recesses 2712, 2714, 2716, 2718 can be located between a center of the depression 2708 and an anterior side of the inferior component 2700. At least two of the motion limiter recesses 2712, 2714, 2716, 2718 can be located between the center of the depression 2708 and a posterior side of the inferior component 2700. Further, at least two of the motion limiter recesses 2712, 2714, 2716, 2718 can be located between the center of the depression 2708 and a first lateral side. Also, at least two of the motion limiter recesses 2712, 2714, 2716, 2718 can be located between the center of the depression 2708 and a second lateral side.

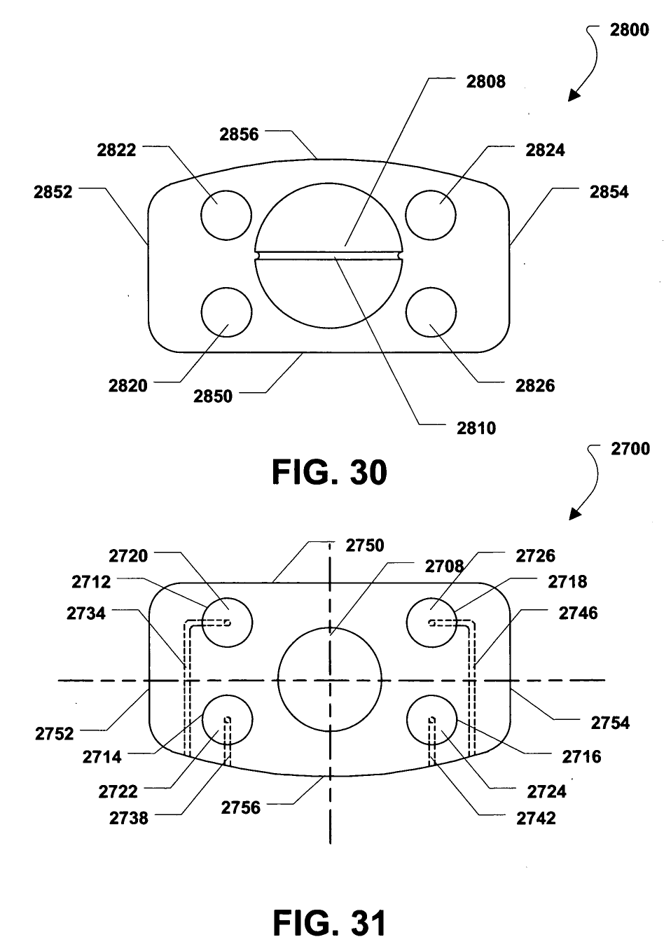

As further illustrated in FIG. 30, the inferior component 2700 includes a first expandable motion limiter 2720 that can be affixed, or otherwise disposed, within the first motion limiter recess 2712. A second expandable motion limiter 2722 can be affixed, or otherwise disposed, within the second motion limiter recess 2714. A third expandable motion limiter 2724 can be affixed, or otherwise disposed, within the third motion limiter recess 2716. Additionally, a fourth expandable motion limiter 2726 can be affixed, or otherwise attached, to the fourth motion limiter recess 2718.

FIG. 26 through FIG. 29 indicate that each of the expandable motion limiters 2720, 2722, 2724, 2726 can be inflated from a deflated position 2728 to one of a plurality of intermediate inflated positions up to a maximum inflated position 2730. The expandable motion limiters 2720, 2722, 2724, 2726 can be inflated to different positions, the same positions, or a combination thereof. In a particular embodiment, the expandable motion limiters 2720, 2722, 2724, 2726 can be inflated with an injectable extended use approved medical materials that remain elastic after curing. Further, the injectable extended use approved medical materials can include polymer materials that remain elastic after curing.

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, sterile air, or a combination thereof. In alternative embodiments, the expandable motion limiters can be inflated with one or more of the following: fibroblasts, lipoblasts, chondroblasts, differentiated stem cells, a combination thereof, or another biologic factor which would create a motion limiting tissue when injected into a bioresorbable motion limiting scaffold.

As shown in FIG. 26 through FIG. 31, the inferior support plate 2702 can include a first port 2732 that is in fluid communication with a first fluid channel 2734 that provides fluid communication to the first expandable motion limiter 2720. The first expandable motion limiter 2720 can be inflated with an injectable extended use approved medical material that is delivered to the first expandable motion limiter 2720 via the first port 2732 and the first fluid channel 2734.

As shown, the inferior support plate 2702 can also include a second port 2736 that is in fluid communication with a second fluid channel 2736 that provides fluid communication to the second expandable motion limiter 2722. The second expandable motion limiter 2722 can be inflated with an injectable extended use approved medical material that is delivered to the second expandable motion limiter 2722 via the second port 2736 and the second fluid channel 2736.

FIG. 26 through FIG. 24 also indicate that the inferior support plate 2702 can include a third port 2740 that is in fluid communication with a third fluid channel 2742 that provides fluid communication to the third expandable motion limiter 2724. The third expandable motion limiter 2724 can be inflated with an injectable extended use approved medical material that is delivered to the third expandable motion limiter 2724 via the third port 2740 and the third fluid channel 2742.

The inferior support plate 2702 can also include a fourth port 2744 that is in fluid communication with a fourth fluid channel 2746 that provides fluid communication to the fourth expandable motion limiter 2726. The fourth expandable motion limiter 2726 can be inflated with an injectable extended user approved medical material that is delivered to the fourth expandable motion limiter 2726 via the fourth port 2744 and the fourth fluid channel 2746.

FIG. 26 through FIG. 29 indicate that the inferior component 2700 can include an inferior keel 2748 that extends from inferior bearing surface 2706. After installation, the inferior keel 2748 can at least partially engage a keel groove that can be established within a cortical rim of a vertebra.

In a particular embodiment, as shown in FIG. 31, the inferior component 2700 can be generally rectangular in shape. For example, the inferior component 2700 can have a substantially straight posterior side 2750. A first straight lateral side 2752 and a second substantially straight lateral side 2754 can extend substantially perpendicular from the posterior side 2750 to an anterior side 2756. In a particular embodiment, the anterior side 2756 can curve outward such that the inferior component 2700 is wider through the middle than along the lateral sides 2752, 2754. Further, in a particular embodiment, the lateral sides 2752, 2754 are substantially the same length.

FIG. 26 and FIG. 27 show that the inferior component 2700 includes a first implant inserter engagement hole 2760 and a second implant inserter engagement hole 2762. In a particular embodiment, the implant inserter engagement holes 2760, 2762 are configured to receive respective dowels, or pins, that extend from an implant inserter (not shown) that can be used to facilitate the proper installation of an intervertebral prosthetic disc, e.g., the intervertebral prosthetic disc 2600 shown in FIG. 26 through FIG. 31.

In a particular embodiment, the superior component 2800 includes a superior support plate 2802 that has a superior articular surface 2804 and a superior bearing surface 2806. In a particular embodiment, the superior articular surface 2804 can be generally curved and the superior bearing surface 2806 can be substantially flat. In an alternative embodiment, the superior articular surface 2804 can be flat and at least a portion of the superior bearing surface 2806 can be curved.

In a particular embodiment, after installation, the superior bearing surface 2806 can be in direct contact with vertebral bone, e.g., cortical bone and cancellous bone. Further, the superior bearing surface 2806 can be coated with a bone-growth promoting substance, e.g., a hydroxyapatite coating formed of calcium phosphate. Additionally, the superior bearing surface 2806 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 26 through FIG. 31, a projection 2808 extends from the superior articular surface 2804 of the superior support plate 2802. In a particular embodiment, the projection 2808 is sized and shaped to engage the depression 2708 of the inferior component 2700. In a particular embodiment, the projection 2808 has a hemi-spherical shape. Alternatively, the projection 2808 can have an elliptical shape, a cylindrical shape, or other arcuate shape. Further, the projection 2808 can be formed with a groove 2810.

The superior support plate 2802 can also include a first motion limiting projection 2822, a second motion limiting projection 2824, a third motion limiting engagement 2826, and a fourth motion limiting projection 2828. In a particular embodiment, the motion limiting projections 2820, 2822, 2824, 2826 are arranged radially around the depression 2808, e.g., in a pattern that mirrors the pattern of the expandable motion limiters 2720, 2722, 2724, 2726. Further, each motion limiting projection 2820, 2822, 2824, 2826 is sized, shaped, and positioned to contact a corresponding expandable motion limiter 2720, 2722, 2724, 2726.

In a particular embodiment, each expandable motion limiter 2720, 2722, 2724, 2726 cooperates with a respective motion limiting projection 2820, 2822, 2824, 2826 in order to limit the motion of the inferior component 2700 with respect to the superior component 2800. For example, by inflating two expandable motion limiters on one side of the depression 2708, a surgeon is able to limit flexion on that side of the depression 2708 and as such, limit the relative motion of the inferior component 2700 with respect to the superior component 2800. Further, this allows the surgeon to limit the motion of a superior vertebra with respect to an inferior vertebra.

The flexibility to alter the range of motion of the intervertebral prosthetic device 2600 provided by the expandable motion limiters 2720, 2722, 2724, 2726 can allow a surgeon to compensate for a deformity in the segment of the spinal column that includes, or is adjacent to, the superior vertebra and inferior vertebra in question. For example, if a patient's spine is curved in a particular direction, one or more motion limiters 2720, 2722, 2724, 2726 opposite the curvature can be inflated to compensate for the curvature. Before or during the surgery, the surgeon can determine any spinal deformity using an X-Ray device, a fluoroscopy device, a computed tomography (CT) device, or any other similar device well known in the art.

FIG. 26 through FIG. 29 indicate that the superior component 2800 can include a keel 2848 that extends from superior bearing surface 2806. After installation, the keel 2848 can at least partially engage a keel groove that can be established within a cortical rim of a vertebra.

As illustrated in FIG. 30, the superior component 2800 can be shaped to match the shape of the inferior component 2700, shown in FIG. 31. Further, the superior component 2800 can be generally rectangular in shape. For example, the superior component 2800 can have a substantially straight posterior side 2850. A first straight lateral side 2852 and a second substantially straight lateral side 2854 can extend substantially perpendicular from the posterior side 2850 to an anterior side 2856. In a particular embodiment, the anterior side 2856 can curve outward such that the superior component 2800 is wider through the middle than along the lateral sides 2852, 2854. Further, in a particular embodiment, the lateral sides 2852, 2854 are substantially the same length.

FIG. 26 and FIG. 27 show that the superior component 2800 includes a first implant inserter engagement hole 2860 and a second implant inserter engagement hole 2862. In a particular embodiment, the implant inserter engagement holes 2860, 2862 are configured to receive respective dowels, or pins, that extend from an implant inserter (not shown) that can be used to facilitate the proper installation of an intervertebral prosthetic disc, e.g., the intervertebral prosthetic disc 2600 shown in FIG. 26 through FIG. 31.

In a particular embodiment, the overall height of the intervertebral prosthetic device 2600 can be in a range from fourteen millimeters to forty-six millimeters (14-46 mm). Further, the installed height of the intervertebral prosthetic device 2600 can be in a range from eight millimeters to sixteen millimeters (8-16 mm). In a particular embodiment, the installed height can be substantially equivalent to the distance between an inferior vertebra and a superior vertebra when the intervertebral prosthetic device 2600 is installed there between.

In a particular embodiment, the length of the intervertebral prosthetic device 2600, e.g., along a longitudinal axis, can be in a range from thirty millimeters to forty millimeters (30-40 mm). Additionally, the width of the intervertebral prosthetic device 2600, e.g., along a lateral axis, can be in a range from twenty-five millimeters to forty millimeters (25-40 mm). Moreover, in a particular embodiment, each keel 2748, 2848 can have a height in a range from three millimeters to fifteen millimeters (3-15 mm).

Although depicted in the Figures as a two piece-design, in alternative embodiments, multiple-piece designs can be employed. For example, in an alternative embodiment, the projection 2808 is not fixed or unitary with either of the support plates 2702, 2802 and, instead, is configured as a substantially rigid spherical member (not shown) that can independently articulate with each support plate 2702, 2802. Additionally or alternatively, each component can comprise multiple components (not shown). These components can articulate with or be fixed to the support plates 2702, 2802. Furthermore, expandable motion limiters can be configured to limit relative motion between any of the components described above or among multiple components.

### Description of a Fifth Embodiment of an Intervertebral Prosthetic Disc

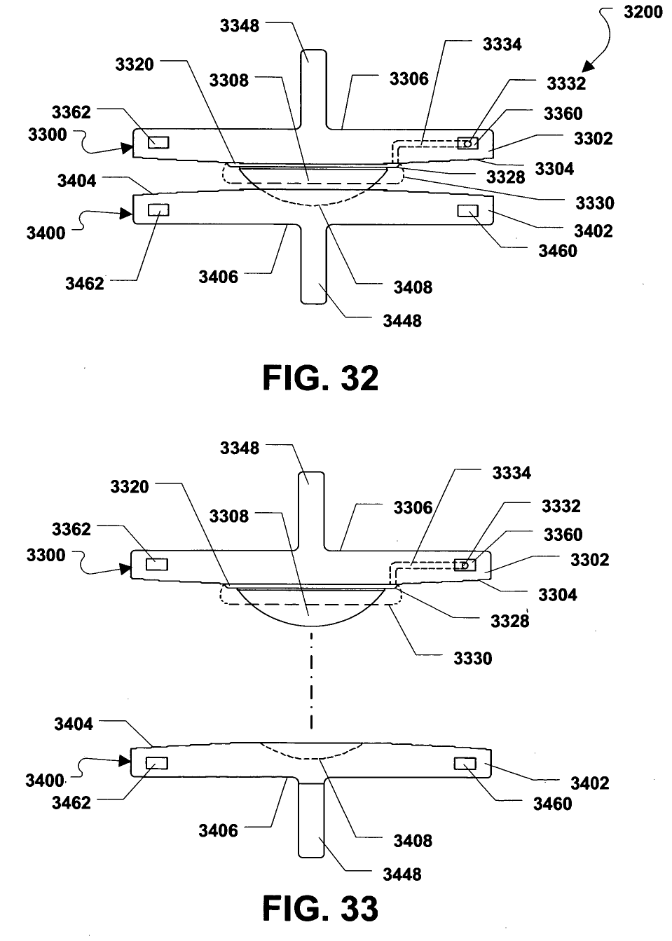

Referring to FIGS. 32 through 37 a fifth embodiment of an intervertebral prosthetic disc is shown and is generally designated 3200. As illustrated, the intervertebral prosthetic disc 3200 includes a superior component 3300 and an inferior component 3400. In a particular embodiment, the components 3300, 3400 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 3300, 3400 can be made from any other substantially rigid biocompatible materials.

In a particular embodiment, the superior component 3300 includes a superior support plate 3302 that has a superior articular surface 3304 and a superior bearing surface 3306. In a particular embodiment, the superior articular surface 3304 can be generally curved and the superior bearing surface 3306 can be substantially flat. In an alternative embodiment, the superior articular surface 3304 can be substantially flat and at least a portion of the superior bearing surface 3306 can be generally curved.

In a particular embodiment, after installation, the superior bearing surface 3306 can be in direct contact with vertebral bone, e.g., cortical bone and cancellous bone. Further, the superior bearing surface 3306 can be coated with a bone-growth promoting substance, e.g., a hydroxyapatite coating formed of calcium phosphate. Additionally, the superior bearing surface 3306 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 32 through FIG. 37, a projection 3308 extends from the superior articular surface 3304 of the superior support plate 3302. In a particular embodiment, the projection 3308 has a hemi-spherical shape. Alternatively, the projection 3308 can have an elliptical shape, a cylindrical shape, or other arcuate shape. Moreover, the projection 3308 can be formed with a groove 3310.

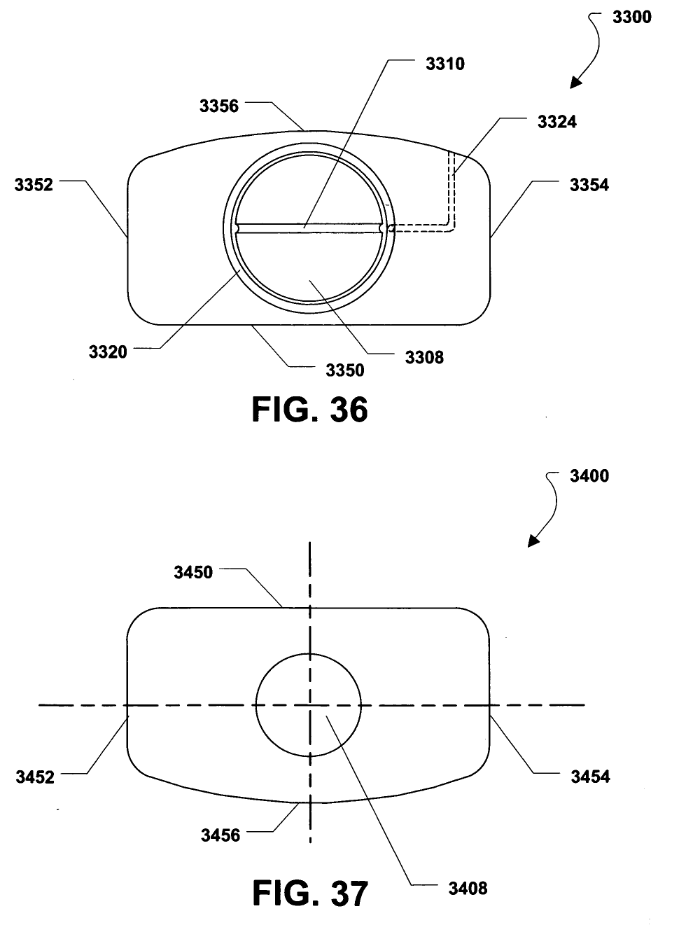

As further illustrated in FIG. 36, the superior component 3300 includes an expandable motion limiter 3320 that is affixed, or otherwise attached to, the superior articular surface 3304. In a particular embodiment, as depicted in FIG. 36, the expandable motion limiter 3320 is generally circular and surrounds the projection 3308. Alternatively, the expandable motion limiter 3320 can be generally elliptical or any other arcuate shape.

FIG. 32 through FIG. 35 indicate that the expandable motion limiter 3320 can be inflated from a deflated position 3328 to one of a plurality of intermediate inflated positions up to a maximum inflated position 3330. In a particular embodiment, the expandable motion limiters 3320 can be inflated with one or more injectable extended use approved medical materials that remain elastic after curing. Further, the injectable extended use approved medical materials can include polymer materials that remain elastic after curing

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, sterile air, or a combination thereof. In alternative embodiments, the expandable motion limiters can be inflated with one or more of the following: fibroblasts, lipoblasts, chondroblasts, differentiated stem cells or other biologic factor which would create a motion limiting tissue when injected into a bioresorbable motion limiting scaffold.

As shown in FIG. 32 through FIG. 36, the superior support plate 3302 can include a port 3332 that is in fluid communication with a fluid channel 3334 that provides fluid communication to the expandable motion limiter 3320. The expandable motion limiter 3320 can be inflated with an injectable extended use approved medical material that is delivered to the expandable motion limiter 3320 via the port 3332 and the fluid channel 3334.

FIG. 32 through FIG. 35 indicate that the superior component 3300 can include a superior keel 3348 that extends from superior bearing surface 3306. During installation, described below, the superior keel 3348 can at least partially engage a keel groove that can be established within a cortical rim of a vertebra.

As illustrated in FIG. 36, the superior component 3300 can be generally rectangular in shape. For example, the superior component 3300 can have a substantially straight posterior side 3350. A first straight lateral side 3352 and a second substantially straight lateral side 3354 can extend substantially perpendicular from the posterior side 3350 to an anterior side 3356. In a particular embodiment, the anterior side 3356 can curve outward such that the superior component 3300 is wider through the middle than along the lateral sides 3352, 3354. Further, in a particular embodiment, the lateral sides 3352, 3354 are substantially the same length.

FIG. 32 and FIG. 33 show that the superior component 3300 includes a first implant inserter engagement hole 3360 and a second implant inserter engagement hole 3362. In a particular embodiment, the implant inserter engagement holes 3360, 3362 are configured to receive respective dowels, or pins, that extend from an implant inserter (not shown) that can be used to facilitate the proper installation of an intervertebral prosthetic disc, e.g., the intervertebral prosthetic disc 3200 shown in FIG. 32 through FIG. 37.

In a particular embodiment, the inferior component 3400 includes an inferior support plate 3402 that has an inferior articular surface 3404 and an inferior bearing surface 3406. In a particular embodiment, the inferior articular surface 3404 can be generally curved and the inferior bearing surface 3406 can be substantially flat. In an alternative embodiment, the inferior articular surface 3404 can be substantially flat and at least a portion of the inferior bearing surface 3406 can be generally curved.

In a particular embodiment, after installation, the inferior bearing surface 3406 can be in direct contact with vertebral bone, e.g., cortical bone and cancellous bone. Further, the inferior bearing surface 3406 can be coated with a bone-growth promoting substance, e.g., a hydroxyapatite coating formed of calcium phosphate. Additionally, the inferior bearing surface 3406 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 32 through FIG. 35, a depression 3408 extends into the inferior articular surface 3404 of the inferior support plate 3402. In a particular embodiment, the depression 3408 is sized and shaped to receive the projection 3308 of the superior component 3300. For example, the depression 3408 can have a hemi-spherical shape. Alternatively, the depression 3408 can have an elliptical shape, a cylindrical shape, or other arcuate shape.

FIG. 32 through FIG. 35 indicate that the inferior component 3400 can include an inferior keel 3448 that extends from inferior bearing surface 3406. During installation, described below, the inferior keel 3448 can at least partially engage a keel groove that can be established within a cortical rim of a vertebra, e.g., the keel groove 350 shown in FIG. 3.

In a particular embodiment, as shown in FIG. 37, the inferior component 3400 can be shaped to match the shape of the superior component 3300, shown in FIG. 36. Further, the inferior component 3400 can be generally rectangular in shape. For example, the inferior component 3400 can have a substantially straight posterior side 3450. A first straight lateral side 3452 and a second substantially straight lateral side 3454 can extend substantially perpendicular from the posterior side 3450 to an anterior side 3456. In a particular embodiment, the anterior side 3456 can curve outward such that the inferior component 3400 is wider through the middle than along the lateral sides 3452, 3454. Further, in a particular embodiment, the lateral sides 3452, 3454 are substantially the same length.

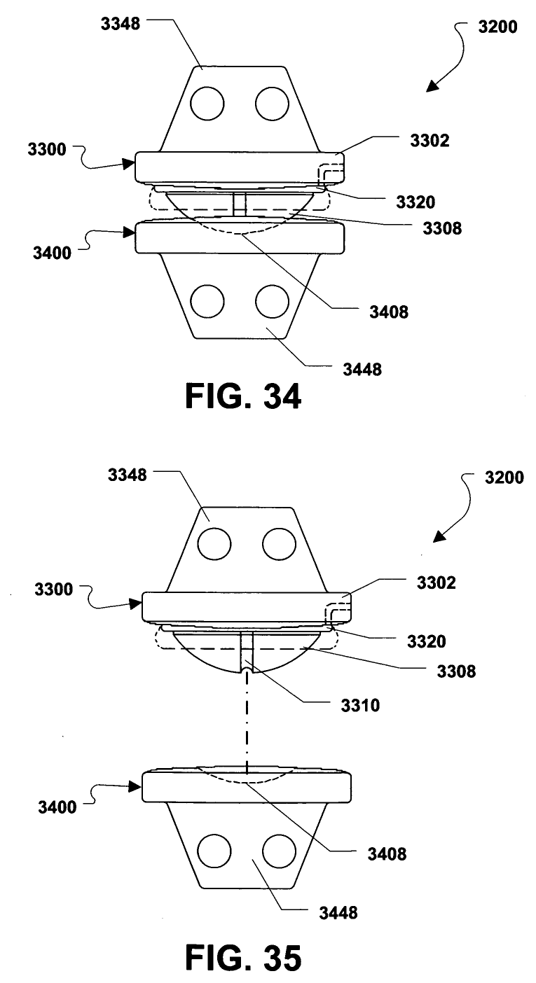

FIG. 32 and FIG. 34 show that the inferior component 3400 includes a first implant inserter engagement hole 3460 and a second implant inserter engagement hole 3462. In a particular embodiment, the implant inserter engagement holes 3460, 3462 are configured to receive respective dowels, or pins, that extend from an implant inserter (not shown) that can be used to facilitate the proper installation of an intervertebral prosthetic disc, e.g., the intervertebral prosthetic disc 3200 shown in FIG. 32 through FIG. 37.

In a particular embodiment, the overall height of the intervertebral prosthetic device 3200 can be in a range from fourteen millimeters to forty-six millimeters (14-46 mm). Further, the installed height of the intervertebral prosthetic device 3200 can be in a range from eight millimeters to sixteen millimeters (8-16 mm). In a particular embodiment, the installed height can be substantially equivalent to the distance between an inferior vertebra and a superior vertebra when the intervertebral prosthetic device 3200 is installed there between.

In a particular embodiment, the length of the intervertebral prosthetic device 3200, e.g., along a longitudinal axis, can be in a range from thirty millimeters to forty millimeters (30-40 mm). Additionally, the width of the intervertebral prosthetic device 3200, e.g., along a lateral axis, can be in a range from twenty-five millimeters to forty millimeters (25-40 mm). Moreover, in a particular embodiment, each keel 3348, 3448 can have a height in a range from three millimeters to fifteen millimeters (3-15 mm).

Although depicted in the Figures as a two piece-design, in alternative embodiments, multiple-piece designs can be employed. For example, in an alternative embodiment, the projection 3308 is not fixed or unitary with either of the support plates 3302, 3402 and, instead, is configured as a substantially rigid spherical member (not shown) that can independently articulate with each support plate 3302, 3402. Additionally or alternatively, each component can comprise multiple components (not shown). These components can articulate with or be fixed to the support plates 3302, 3402. Furthermore, expandable motion limiters can be configured to limit relative motion between any of the components described above or among multiple components.

### Description of a Sixth Embodiment of an Intervertebral Prosthetic Disc

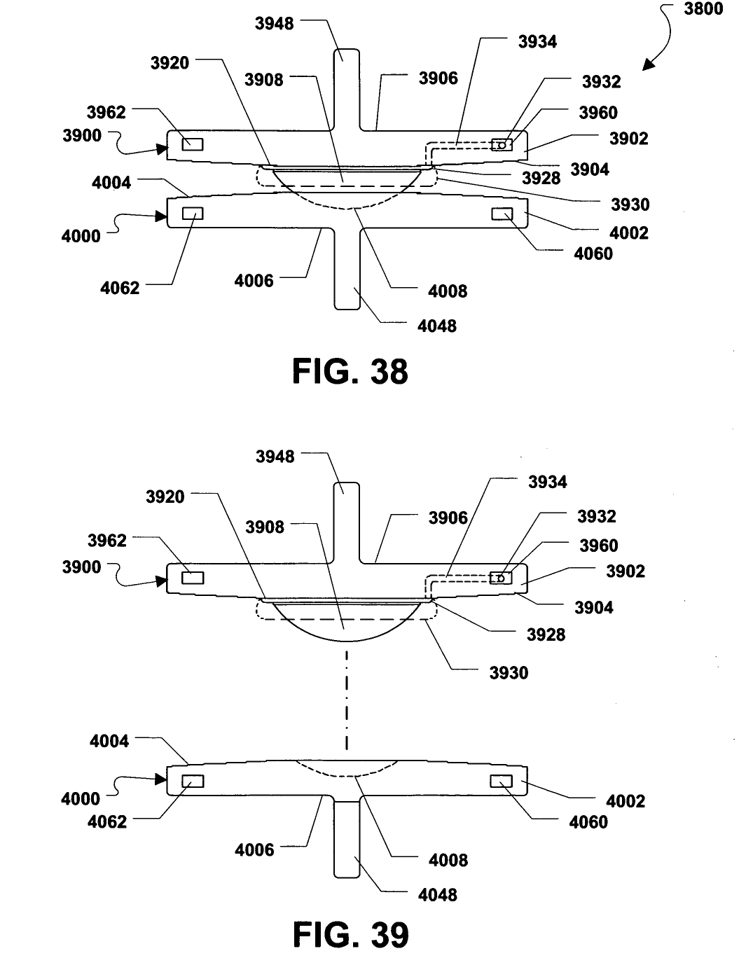

Referring to FIGS. 38 through 43 a sixth embodiment of an intervertebral prosthetic disc is shown and is generally designated 3800. As illustrated, the intervertebral prosthetic disc 3800 includes a superior component 3900 and an inferior component 4000. In a particular embodiment, the components 3900, 4000 can be made from one or more extended use approved medical materials. For example, the materials can be metal containing materials, polymer materials, or composite materials that include metals, polymers, or combinations of metals and polymers.

In a particular embodiment, the metal containing materials can be metals. Further, the metal containing materials can be ceramics. Also, the metals can be pure metals or metal alloys. The pure metals can include titanium. Moreover, the metal alloys can include stainless steel, a cobalt-chrome-molybdenum alloy, e.g., ASTM F-999 or ASTM F-75, a titanium alloy, or a combination thereof.

The polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Alternatively, the components 3900, 4000 can be made from any other substantially rigid biocompatible materials.

In a particular embodiment, the superior component 3900 includes a superior support plate 3902 that has a superior articular surface 3904 and a superior bearing surface 3906. In a particular embodiment, the superior articular surface 3904 can be generally curved and the superior bearing surface 3906 can be substantially flat. In an alternative embodiment, the superior articular surface 3904 can be substantially flat and at least a portion of the superior bearing surface 3906 can be generally curved.

In a particular embodiment, after installation, the superior bearing surface 3906 can be in direct contact with vertebral bone, e.g., cortical bone and cancellous bone. Further, the superior bearing surface 3906 can be coated with a bone-growth promoting substance, e.g., a hydroxyapatite coating formed of calcium phosphate. Additionally, the superior bearing surface 3906 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 38 through FIG. 43, a projection 3908 extends from the superior articular surface 3904 of the superior support plate 3902. In a particular embodiment, the projection 3908 has a hemi-spherical shape. Alternatively, the projection 3908 can have an elliptical shape, a cylindrical shape, or other arcuate shape. Moreover, the projection 3908 can be formed with a groove 3910.

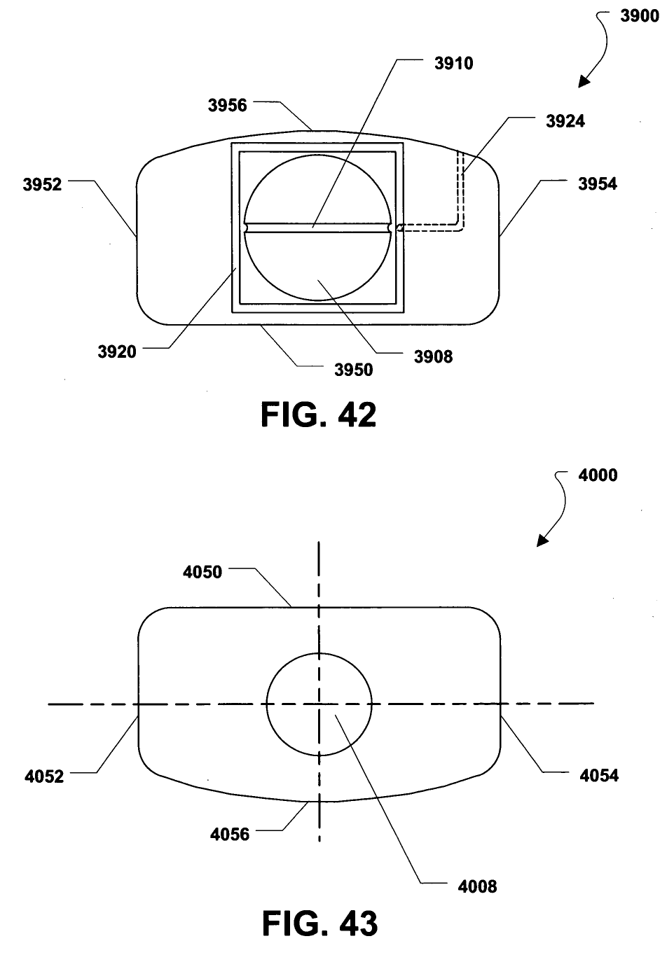

As further illustrated in FIG. 42, the superior component 3900 includes an expandable motion limiter 3920 that is affixed, or otherwise attached to, the superior articular surface 3904. In a particular embodiment, as depicted in FIG. 42, the expandable motion limiter 3920 is generally square and surrounds the projection 3908. Alternatively, the expandable motion limiter 3920 can be generally rectangular or any other polygonal shape.

FIG. 38 through FIG. 41 indicate that the expandable motion limiter 3920 can be inflated from a deflated position 3928 to one of a plurality of intermediate inflated positions up to a maximum inflated position 3930. In a particular embodiment, the expandable motion limiters 3920 can be inflated with one or more injectable extended use approved medical materials that remain elastic after curing. Further, the injectable extended use approved medical materials can include polymer materials that remain elastic after curing

For example, the polymer materials can include polyurethane materials, polyolefin materials, polyether materials, silicone materials, or a combination thereof. Further, the polyolefin materials can include polypropylene, polyethylene, halogenated polyolefin, flouropolyolefin, or a combination thereof. The polyether materials can include polyetherketone (PEK), polyetheretherketone (PEEK), polyetherketoneketone (PEKK), polyaryletherketone (PAEK), or a combination thereof. Also, the silicone materials can include a silicone hydrogel.

In an alternative embodiment, the injectable extended use approved medical materials can include one or more fluids such as sterile water, saline, sterile air, or a combination thereof. In alternative embodiments, the expandable motion limiters can be inflated with one or more of the following: fibroblasts, lipoblasts, chondroblasts, differentiated stem cells or other biologic factor which would create a motion limiting tissue when injected into a bioresorbable motion limiting scaffold.

As shown in FIG. 38 through FIG. 42, the superior support plate 3902 can include a port 3932 that is in fluid communication with a fluid channel 3934 that provides fluid communication to the expandable motion limiter 3920. The expandable motion limiter 3920 can be inflated with an injectable extended use approved medical material that is delivered to the expandable motion limiter 3920 via the port 3932 and the fluid channel 3934.

FIG. 38 through FIG. 41 indicate that the superior component 3900 can include a superior keel 3948 that extends from superior bearing surface 3906. During installation, described below, the superior keel 3948 can at least partially engage a keel groove that can be established within a cortical rim of a vertebra.

As illustrated in FIG. 42, the superior component 3900 can be generally rectangular in shape. For example, the superior component 3900 can have a substantially straight posterior side 3950. A first straight lateral side 3952 and a second substantially straight lateral side 3954 can extend substantially perpendicular from the posterior side 3950 to an anterior side 3956. In a particular embodiment, the anterior side 3956 can curve outward such that the superior component 3900 is wider through the middle than along the lateral sides 3952, 3954. Further, in a particular embodiment, the lateral sides 3952, 3954 are substantially the same length.

FIG. 38 and FIG. 39 show that the superior component 3900 includes a first implant inserter engagement hole 3960 and a second implant inserter engagement hole 3962. In a particular embodiment, the implant inserter engagement holes 3960, 3962 are configured to receive respective dowels, or pins, that extend from an implant inserter (not shown) that can be used to facilitate the proper installation of an intervertebral prosthetic disc, e.g., the intervertebral prosthetic disc 3800 shown in FIG. 38 through FIG. 43.

In a particular embodiment, the inferior component 4000 includes an inferior support plate 4002 that has an inferior articular surface 4004 and an inferior bearing surface 4006. In a particular embodiment, the inferior articular surface 4004 can be generally curved and the inferior bearing surface 4006 can be substantially flat. In an alternative embodiment, the inferior articular surface 4004 can be substantially flat and at least a portion of the inferior bearing surface 4006 can be generally curved.

In a particular embodiment, after installation, the inferior bearing surface 4006 can be in direct contact with vertebral bone, e.g., cortical bone and cancellous bone. Further, the inferior bearing surface 4006 can be coated with a bone-growth promoting substance, e.g., a hydroxyapatite coating formed of calcium phosphate. Additionally, the inferior bearing surface 4006 can be roughened prior to being coated with the bone-growth promoting substance to further enhance bone on-growth. In a particular embodiment, the roughening process can include acid etching; knurling; application of a bead coating, e.g., cobalt chrome beads; application of a roughening spray, e.g., titanium plasma spray (TPS); laser blasting; or any other similar process or method.

As illustrated in FIG. 38 through FIG. 41, a depression 4008 extends into the inferior articular surface 4004 of the inferior support plate 4002. In a particular embodiment, the depression 4008 is sized and shaped to receive the projection 3908 of the superior component 3900. For example, the depression 4008 can have a hemi-spherical shape. Alternatively, the depression 4008 can have an elliptical shape, a cylindrical shape, or other arcuate shape.

FIG. 38 through FIG. 41 indicate that the inferior component 4000 can include an inferior keel 4048 that extends from inferior bearing surface 4006. During installation, described below, the inferior keel 4048 can at least partially engage a keel groove that can be established within a cortical rim of a vertebra, e.g., the keel groove 410 shown in FIG. 3.

In a particular embodiment, as shown in FIG. 43, the inferior component 4000 can be shaped to match the shape of the superior component 3900, shown in FIG. 42. Further, the inferior component 4000 can be generally rectangular in shape. For example, the inferior component 4000 can have a substantially straight posterior side 4050. A first straight lateral side 4052 and a second substantially straight lateral side 4054 can extend substantially perpendicular from the posterior side 4050 to an anterior side 4056. In a particular embodiment, the anterior side 4056 can curve outward such that the inferior component 4000 is wider through the middle than along the lateral sides 4052, 4054. Further, in a particular embodiment, the lateral sides 4052, 4054 are substantially the same length.

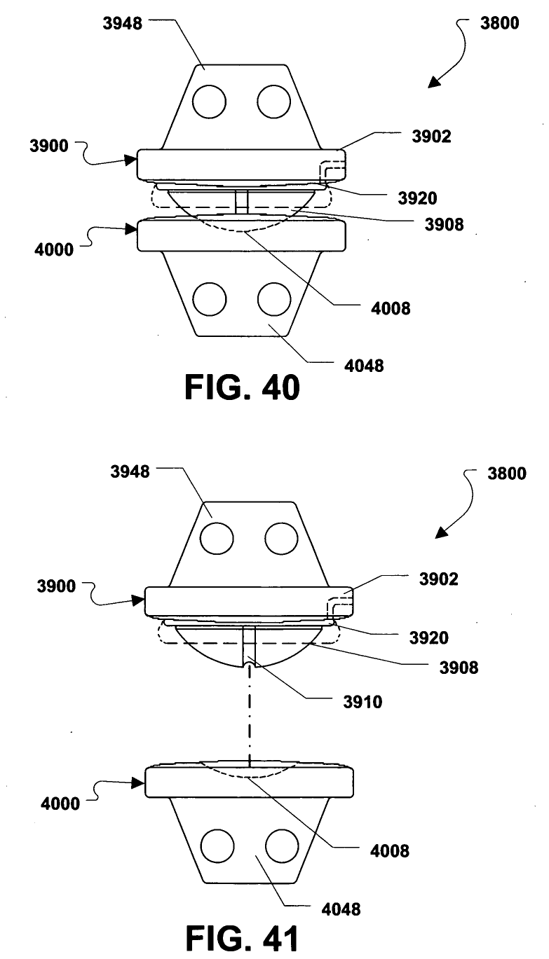

FIG. 38 and FIG. 40 show that the inferior component 4000 includes a first implant inserter engagement hole 4060 and a second implant inserter engagement hole 4062. In a particular embodiment, the implant inserter engagement holes 4060, 4062 are configured to receive respective dowels, or pins, that extend from an implant inserter (not shown) that can be used to facilitate the proper installation of an intervertebral prosthetic disc, e.g., the intervertebral prosthetic disc 3800 shown in FIG. 38 through FIG. 43.

In a particular embodiment, the overall height of the intervertebral prosthetic device 3800 can be in a range from fourteen millimeters to forty-six millimeters (14-46 mm). Further, the installed height of the intervertebral prosthetic device 3800 can be in a range from eight millimeters to sixteen millimeters (8-16 mm). In a particular embodiment, the installed height can be substantially equivalent to the distance between an inferior vertebra and a superior vertebra when the intervertebral prosthetic device 3800 is installed there between.

In a particular embodiment, the length of the intervertebral prosthetic device 3800, e.g., along a longitudinal axis, can be in a range from thirty millimeters to forty millimeters (30-40 mm). Additionally, the width of the intervertebral prosthetic device 3800, e.g., along a lateral axis, can be in a range from twenty-five millimeters to forty millimeters (25-40 mm). Moreover, in a particular embodiment, each keel 3948, 4048 can have a height in a range from three millimeters to fifteen millimeters (3-15 mm).

Although depicted in the Figures as a two piece-design, in alternative embodiments, multiple-piece designs can be employed. For example, in an alternative embodiment, the projection 3908 is not fixed or unitary with either of the support plates 3902, 4002 and, instead, is configured as a substantially rigid spherical member (not shown) that can independently articulate with each support plate 3902, 4002. Additionally or alternatively, each component can comprise multiple components (not shown). These components can articulate with or be fixed to the support plates 3902, 4002. Furthermore, expandable motion limiters can be configured to limit relative motion between any of the components described above or among multiple components.

### Description of a First Embodiment of an Implant Inserter

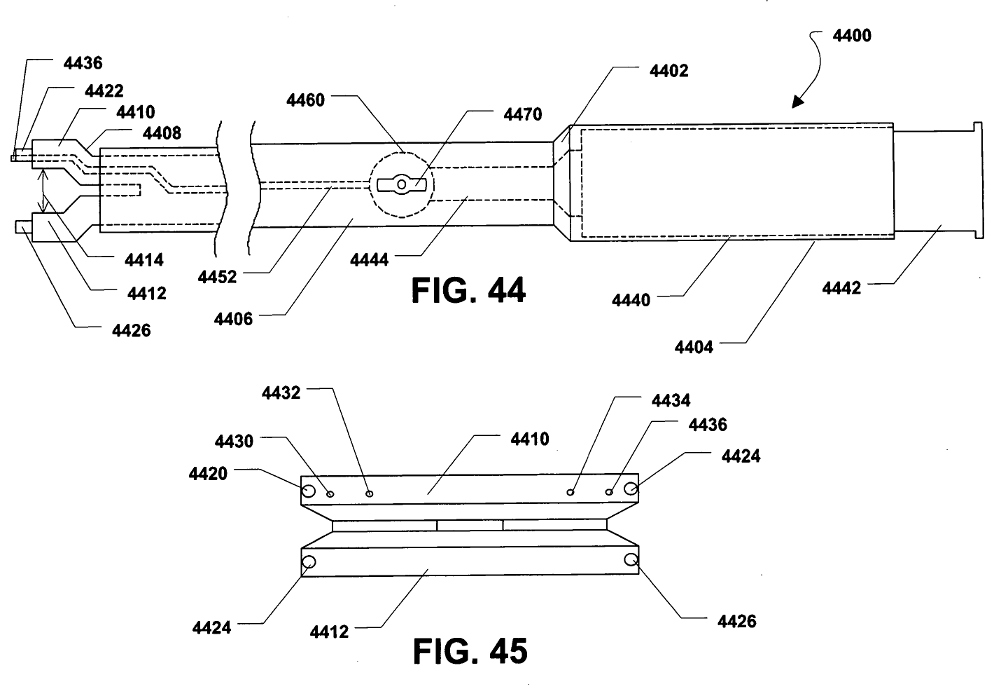

Referring to FIG. 44 through FIG. 48, a first embodiment of an implant inserter is shown and is generally designated 4400. In a particular embodiment the implant inserter 4400 can be used to facilitate installing of an intervertebral prosthetic disc, e.g., an intervertebral prosthetic disc according to one or more of the embodiments described herein.

As shown in FIG. 44, the implant inserter 4400 can include a body 4402. The body 4402 can include a proximal portion 4404 and a distal portion 4406. An implant engagement head 4408 can be affixed to the body 4402, e.g., to the distal portion 4406 of the body 4402. In a particular embodiment, the implant engagement head 4408 can include a superior arm 4410 and an inferior arm 4412. Further, the implant engagement head 4408 can slide relative to the body 4402. As the implant engagement head 4408 moves relative to the body 4402 a distance 4414 between the arms 4410, 4412 can change. For example, as the implant engagement head 4408 slides into the distal portion 4406 of the body 4402, the distance 4414 between the arms 4410, 4412 can decrease. Conversely, as the implant engagement head 4408 slides out of the distal portion 4406 of the body 4402, the distance 4414 between the arms 4410, 4412 can increase.

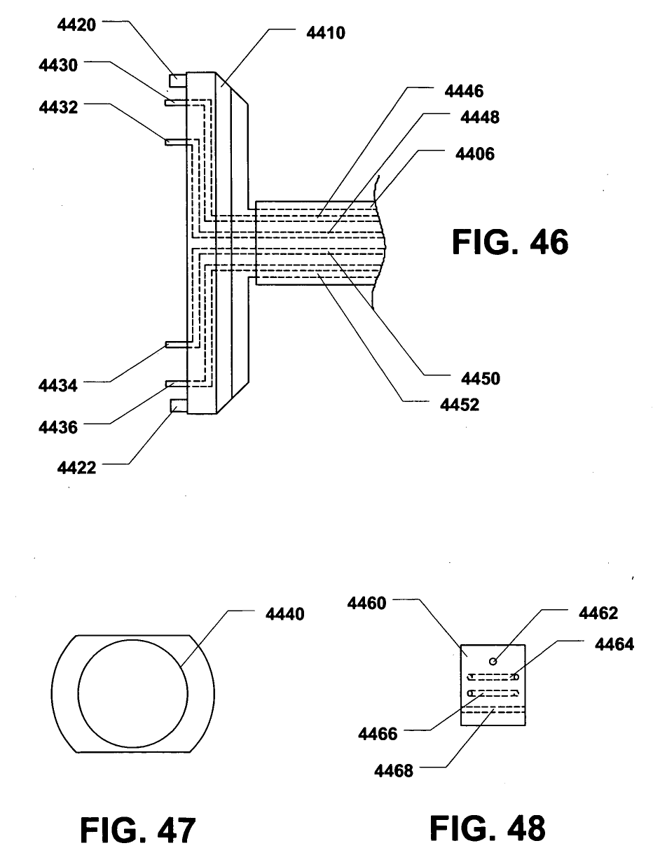

As illustrated in FIG. 45 and FIG. 46, the superior arm 4410 of the implant engagement head 4408 includes a first superior dowel 4420 and a second superior dowel 4422. The inferior arm 4412 of the implant engagement head 4408 includes a first inferior dowel 4424 and a second inferior dowel 4426. As shown the dowels 4420, 4422, 4424, 4426 can be circular. Alternatively, the dowels 4420, 4422, 4424, 4426 can be elliptical, triangular, square, rectangular, or any polygonal shape. In a particular embodiment, the superior arm 4410 of the implant engagement head 4408 also includes a first injector 4430, a second injector 4432, a third injector 4434, and a fourth injector 4436.

In a particular embodiment, as depicted in FIG. 44 and FIG. 47, the proximal portion 4404 of the body 4402 is formed with a generally cylindrical plunger chamber 4440 into which a generally cylindrical plunger 4442 can be inserted. In a particular embodiment, the plunger 4442 can slide relative to the body 4402 within the plunger chamber 4440.

FIG. 44 shows that the body 4402 can be formed with a primary fluid channel 4444 that is in fluid communication with the plunger chamber 4440. Further, the primary fluid channel 4444 can communicate with a first secondary fluid channel 4446, a second secondary fluid channel 4448, a third secondary fluid channel 4450, and a fourth secondary fluid channel 4452. Each of the secondary fluid channels 4446, 4448, 4450, 4452 can communicate with a respective injector 4430, 4432, 4434, 4436. For example, the first secondary fluid channel 4446 can communicate with the first injector 4430, the second secondary fluid channel 4448 can communicate with the second injector 4432, the third secondary fluid channel 4450 can communicate with the third injector 4434, and the fourth secondary fluid channel 4452 can communicate with the fourth injector 4436.

As shown in FIG. 44, a generally cylindrical stop cock 4460 can be installed within the body 4402 between the primary fluid channel 4444 and the secondary fluid channels 4446, 4448, 4450, 4452. Moreover, the stop cock 4460 can be in fluid communication with the primary fluid channel 4444 and the secondary fluid channels 4446, 4448, 4450, 4452 and can control the communication of fluid between the primary fluid channel 4444 and the secondary fluid channels 4446, 4448, 4450, 4452.

In particular, the stop cock 4460 can include a first fluid transfer channel 4462, a second fluid transfer channel 4464, a third fluid transfer channel 4466, and a fourth fluid transfer channel 4468 established radially therethrough. In a particular embodiment, the fluid transfer channels 4462, 4464, 4466, 4468 can be established within the stop cock 4460 so that the secondary fluid channels 4446, 4448, 4450, 4452 can communicate with the primary fluid channel 4444 via the stop cock 4460 individually, i.e., one at a time. For example, the fluid transfer channels 4462, 4464, 4466, 4468 can be established at different locations linearly along the stop cock 4460 and at different radial angles through the stop cock 4460.

In a particular embodiment, the stop cock 4460 can be rotated by turning a knob 4470 that is coupled thereto. As the stop cock 4460 is rotated to one of four fluid transfer positions, a fluid transfer channel 4462, 4464, 4466, 4468 can communicate fluid from the primary fluid channel 4444 to a corresponding secondary fluid channel 4446, 4448, 4450, 4452 and injector 4430, 4432, 4434, 4436. As such, abuser, e.g., a surgeon, can select which injector 4430, 4432, 4434, 4436 can be used to inject a fluid into an expandable motion limiter, e.g., an expandable motion limiter according to one of the embodiments disclosed herein.

### Description of a Second Embodiment of an Implant Inserter

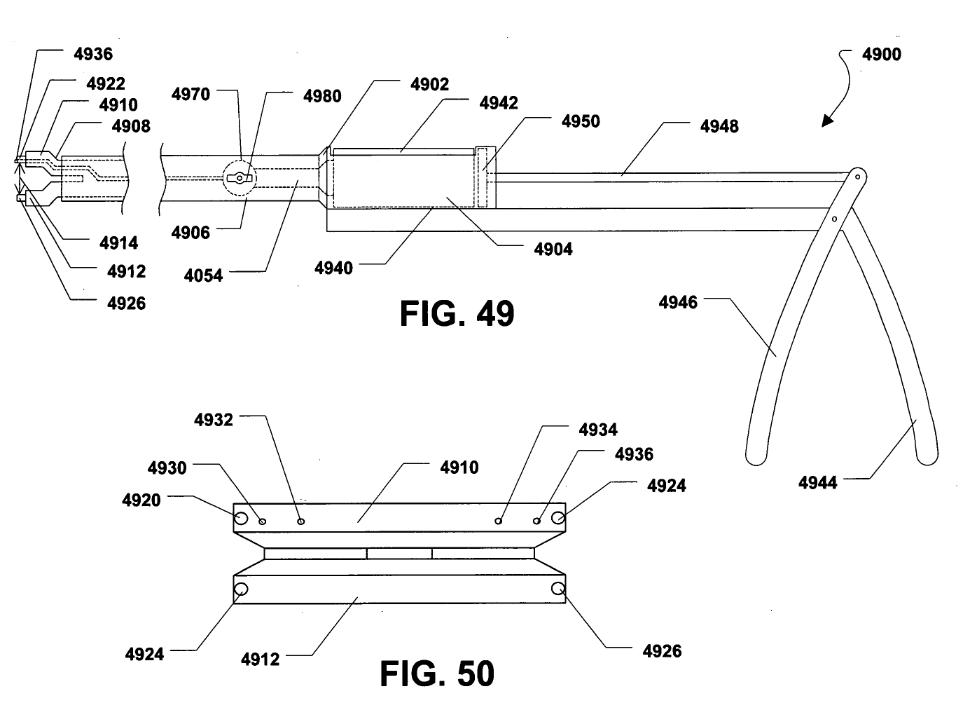

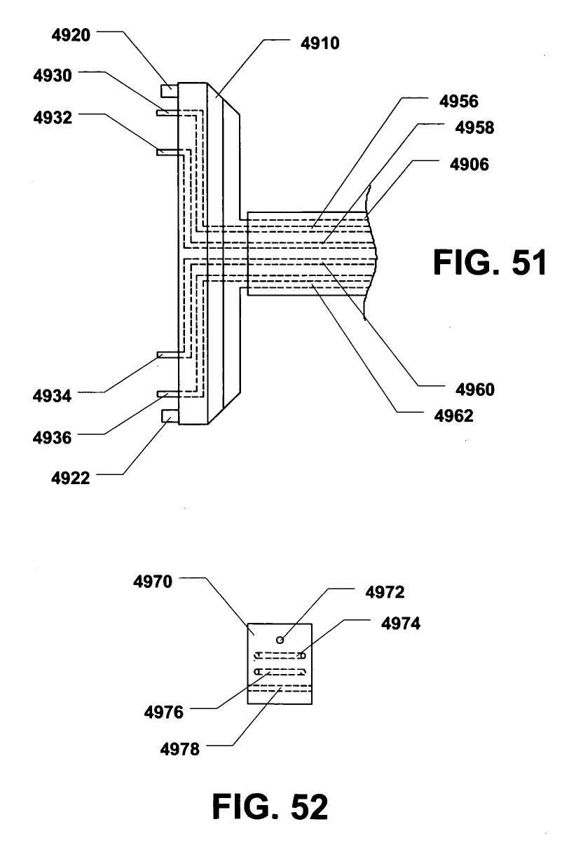

Referring to FIG. 49 through FIG. 52, a second embodiment of an implant inserter is shown and is generally designated 4900. In a particular embodiment the implant inserter 4900 can be used to facilitate installing of an intervertebral prosthetic disc, e.g., an intervertebral prosthetic disc according to one or more of the embodiments described herein.

As shown in FIG. 49, the implant inserter 4900 can include a body 4902. The body 4902 can include a proximal portion 4904 and a distal portion 4906. An implant engagement head 4908 can be affixed to the body 4902, e.g., to the distal portion 4906 of the body 4902. In a particular embodiment, the implant engagement head 4908 can include a superior arm 4910 and an inferior arm 4912. Further, the implant engagement head 4908 can slide relative to the body 4902. As the implant engagement head 4908 moves relative to the body 4902 a distance 4914 between the arms 4910, 4912 can change. For example, as the implant engagement head 4908 slides into the distal portion 4906 of the body 4902, the distance 4914 between the arms 4910, 4912 can decrease. Conversely, as the implant engagement head 4908 slides out of the distal portion 4906 of the body 4902, the distance 4914 between the arms 4910, 4912 can increase.

As illustrated in FIG. 45 and FIG. 46, the superior arm 4910 of the implant engagement head 4908 includes a first superior dowel 4920 and a second superior dowel 4922. The inferior arm 4912 of the implant engagement head 4908 includes a first inferior dowel 4924 and a second inferior dowel 4926. As shown the dowels 4920, 4922, 4924, 4926 can be circular. Alternatively, the dowels 4920; 4922, 4924, 4926 can be elliptical, triangular, square, rectangular, or any polygonal shape. In a particular embodiment, the superior arm 4910 of the implant engagement head 4908 also includes a first injector 4930, a second injector 4932, a third injector 4934, and a fourth injector 4936.

In a particular embodiment, as depicted in FIG. 49, the proximal portion 4904 of the body 4902 is formed with a generally cylindrical cartridge chamber 4940 into which a generally cylindrical cartridge 4942 can be inserted. In a particular embodiment, the cartridge 4942 can be filled with a material that can be used to inflate an expandable motion limiter, e.g., an expandable motion limiter according to one or more of the embodiments described herein.

As illustrated in FIG. 49, a stationary handle 4944 extends from the proximal portion 4904 of the body 4902. Further, a movable handle 4946 can be coupled to the stationary handle 4944. In a particular embodiment, the movable handle 4946 can rotate with respect to the stationary handle 4944. Moreover, the movable handle 4946 can be connected to a plunger arm 4948. A plunger 4950 can be coupled, or otherwise attached, to the end of the plunger arm 4948. In a particular embodiment, the plunger 4950 can be configured to engage the cartridge 4942. Additionally, the movable handle 4946 can be moved toward the stationary handle 4944 to cause the plunger arm 4948 to move toward the cartridge 4942. The plunger 4950 can be configured to slide within the cartridge 4942 and force the material within the cartridge 4942 to exit the cartridge.

FIG. 49 shows that the body 4902 can be formed with a primary fluid channel 4954 that is in fluid communication with the cartridge chamber 4940. Further, the primary fluid channel 4954 can communicate with a first secondary fluid channel 4956, a second secondary fluid channel 4958, a third secondary fluid channel 4960, and a fourth secondary fluid channel 4962. Each of the secondary fluid channels 4956, 4958, 4960, 4962 can communicate with a respective injector 4930, 4932, 4934, 4936. For example, the first secondary fluid channel 4956 can communicate with the first injector 4930, the second secondary fluid channel 4958 can communicate with the second injector 4932, the third secondary fluid channel 4960 can communicate with the third injector 4934, and the fourth secondary fluid channel 4962 can communicate with the fourth injector 4936.

As shown in FIG. 49, a generally cylindrical stop cock 4970 can be installed within the body 4902 between the primary fluid channel 4954 and the secondary fluid channels 4956, 4958, 4960, 4962. Moreover, the stop cock 4970 can be in fluid communication with the primary fluid channel 4954 and the secondary fluid channels 4956, 4958, 4960, 4962 and can control the communication of fluid between the primary fluid channel 4954 and the secondary fluid channels 4956, 4958, 4960, 4962.

In particular, the stop cock 4970 can include a first fluid transfer channel 4972, a second fluid transfer channel 4974, a third fluid transfer channel 4976, and a fourth fluid transfer channel 4978 established radially therethrough. In a particular embodiment, the fluid transfer channels 4972, 4974, 4976, 4978 can be established within the stop cock 4970 so that the secondary fluid channels 4956, 4958, 4960, 4962 can communicate with the primary fluid channel 4954 via the stop cock 4970 individually, i.e., one at a time. For example, the fluid transfer channels 4972, 4974, 4976, 4978 can be established at different locations linearly along the stop cock 4970 and at different radial angles through the stop cock 4970.

In a particular embodiment, the stop cock 4970 can be rotated by turning a knob 4980 that is coupled thereto. As the stop cock 4970 is rotated to one of four fluid transfer positions, a fluid transfer channel 4972, 4974, 4976, 4978 can communicate fluid from the primary fluid channel 4954 to a corresponding secondary fluid channel 4956, 4958, 4960, 4962 and injector 4930, 4932, 4934, 4936. As such, a user, e.g., a surgeon, can select which injector 4930, 4932, 4934, 4936 can be used to inject a fluid into an expandable motion limiter, e.g., an expandable motion limiter according to one of the embodiments disclosed herein.

### Description of a Third Embodiment of an Implant Inserter

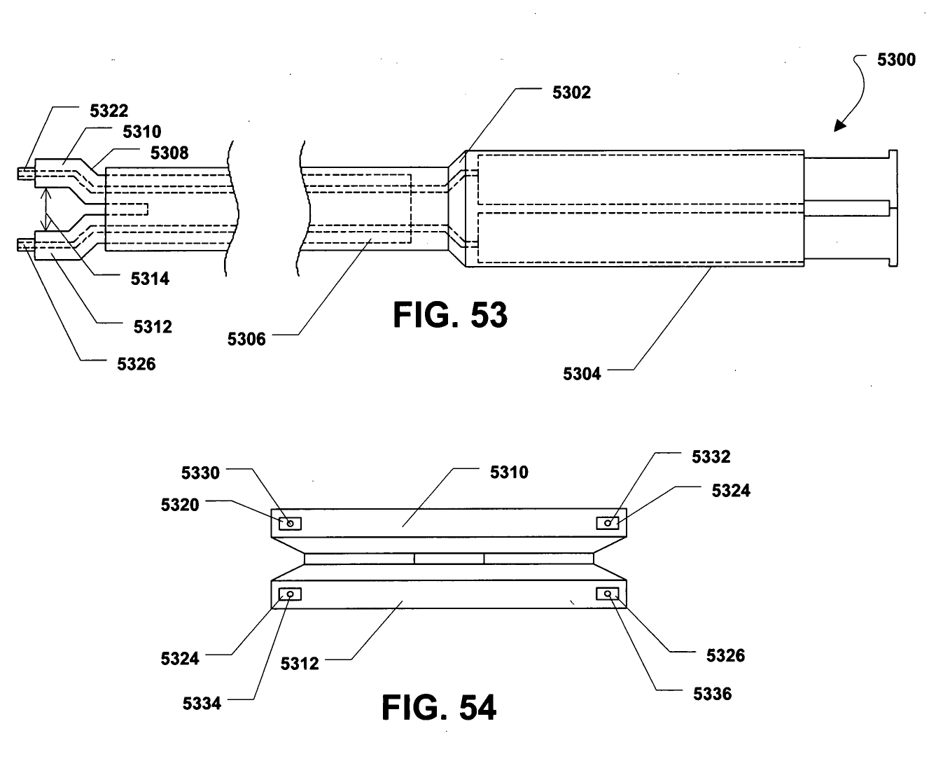

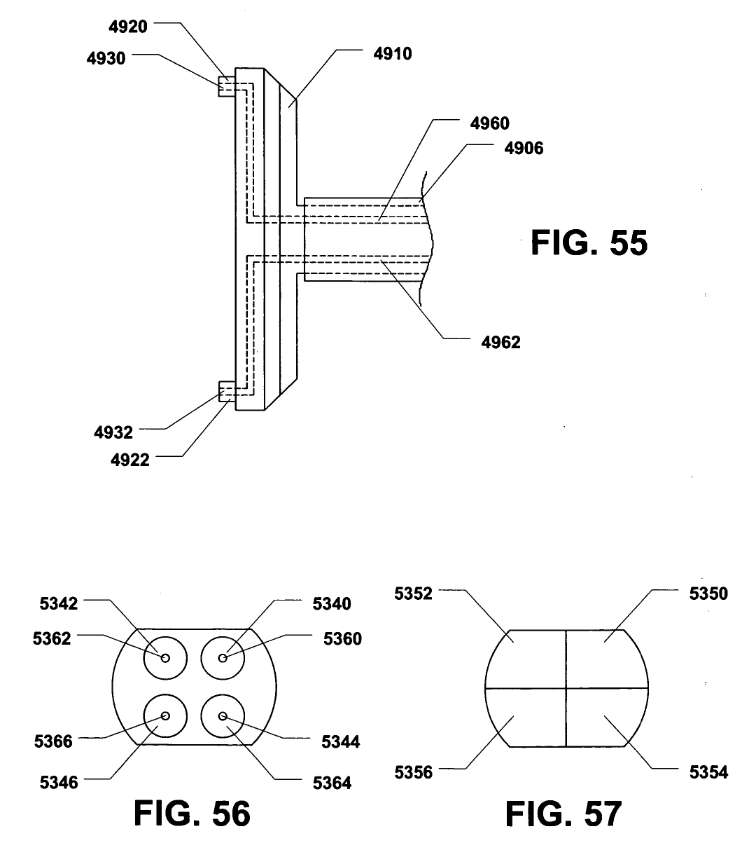

Referring to FIG. 53 through FIG. 57, a third embodiment of an implant inserter is shown and is generally designated 5300. In a particular embodiment the implant inserter 5300 can be used to facilitate installing of an intervertebral prosthetic disc, e.g., an intervertebral prosthetic disc according to one or more of the embodiments described herein.

As shown in FIG. 53, the implant inserter 5300 can include a body 5302. The body 5302 can include a proximal portion 5304 and a distal portion 5306. An implant engagement head 5308 can be affixed to the body 5302, e.g., to the distal portion 5306 of the body 5302. In a particular embodiment, the implant engagement head 5308 can include a superior arm 5310 and an inferior arm 5312. Further, the implant engagement head 5308 can slide relative to the body 5302. As the implant engagement head 5308 moves relative to the body 5302 a distance 5314 between the arms 5310, 5312 can change. For example, as the implant engagement head 5308 slides into the distal portion 5306 of the body 5302, the distance 5314 between the arms 5310, 5312 can decrease. Conversely, as the implant engagement head 5308 slides out of the distal portion 5306 of the body 5302, the distance 5314 between the arms 5310, 5312 can increase.

As illustrated in FIG. 54, the superior arm 5310 of the implant engagement head 5308 includes a first superior dowel 5320 and a second superior dowel 5322. The inferior arm 5312 of the implant engagement head 5308 includes a first inferior dowel 5324 and a second inferior dowel 5326. As shown, the dowels 5320, 5322, 5324, 5326 can be rectangular. Alternatively, the dowels 5320, 5322, 5324, 5326 can be triangular, square, circular, elliptical, or any polygonal shape.

FIG. 54 indicates that the superior arm 5310 of the implant engagement head 5308 can also include a first superior injector 5330 and a second superior injector 5332. Further, the inferior arm 5312 of the implant engagement head 5309 can include a first inferior injector 5334 and a second inferior injector 5336. In a particular embodiment, each injector 5330, 5332, 5334, 5336 can extend through a respective dowel 5320, 5322, 5324, 5326.

In a particular embodiment, as depicted in FIG. 56, the proximal portion 5304 of the body 5302 is formed with a generally cylindrical first superior plunger chamber 5340, a generally cylindrical second superior plunger chamber 5342, a generally cylindrical first inferior plunger chamber 5344, and a generally cylindrical second inferior plunger chamber 5346. Moreover, as shown in FIG. 57, a generally cylindrical first superior plunger 5350 can be inserted into the first superior plunger chamber 5340. A generally cylindrical second superior plunger 5352 can be inserted into the second superior plunger chamber 5342. A generally cylindrical first inferior plunger 5354 can be inserted into the first inferior plunger chamber 5344. Also, a generally cylindrical second inferior plunger 5356 can be inserted into the second inferior plunger chamber 5346. In a particular embodiment, each plunger 5350, 5352, 5354, 5356 can slide relative to the body 5302 within a respective plunger chamber 5340, 5342, 5344, 5346.

FIG. 56 shows that the body 5302 can be formed with a first superior fluid channel 5360 that is in fluid communication with the first superior plunger chamber 5340. Further, the first superior fluid channel 5360 can communicate with the first superior injector 5330. The body 5302 can also be formed with a second superior fluid channel 5362 that is in fluid communication with the second superior plunger chamber 5342. The second superior fluid channel 5360 can communicate with the second superior injector 5332.

In a particular embodiment, the body 5302 can be formed with a first inferior fluid channel 5364 that is in fluid communication with the first inferior plunger chamber 5344. Further, the first inferior fluid channel 5364 can communicate with the first inferior injector 5334. The body 5302 can also be formed with a fourth inferior fluid channel 5366 that is in fluid communication with the second inferior plunger chamber 5346. The second inferior fluid channel 5366 can communicate with the second inferior injector 5336.

During use, a user, e.g., a surgeon, can select which injector 5330, 5332, 5334, 5336 can be used to inject a fluid into an expandable motion limiter, e.g., an expandable motion limiter according to one of the embodiments disclosed herein, by selecting a corresponding plunger 5350, 5352, 5354, 5356. The selected plunger 5350, 5353, 5354, 5356 can be slid into the corresponding plunger chamber 5350, 5342, 5344, 5346 in order to force material from within the plunger chamber 5350, 5342, 5344, 5346 to travel through the fluid channel 5360, 5362, 5364, 5366 and exit through the selected injector 5330, 5332, 5334, 5336.

### Description of a Fourth Embodiment of an Implant Inserter

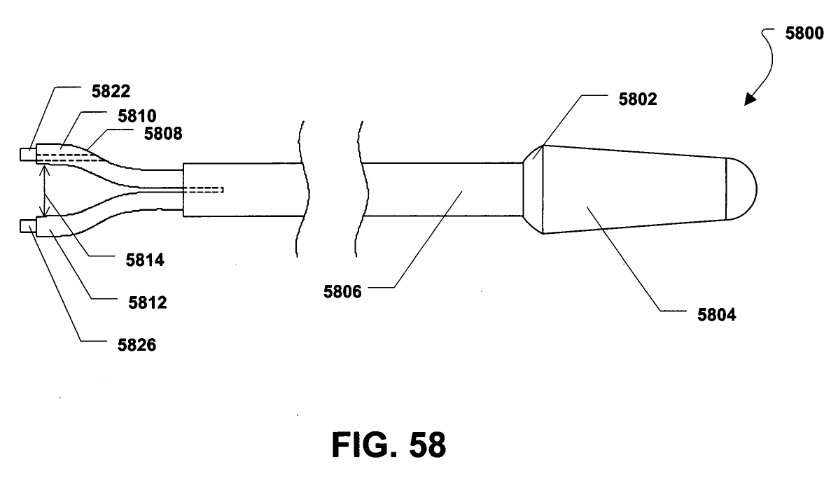

Referring to FIG. 58 through FIG. 60, a third embodiment of an implant inserter is shown and is generally designated 5800. In a particular embodiment the implant inserter 5800 can be used to facilitate installing of an intervertebral prosthetic disc, e.g., an intervertebral prosthetic disc according to one or more of the embodiments described herein.

As shown in FIG. 58, the implant inserter 5800 can include a body 5802. The body 5802 can include a proximal portion 5804 and a distal portion 5806. An implant engagement head 5808 can be affixed to the body 5802, e.g., to the distal portion 5806 of the body 5802. In a particular embodiment, the implant engagement head 5808 can include a superior arm 5810 and an inferior arm 5812. Further, the implant engagement head 5808 can slide relative to the body 5802. As the implant engagement head 5808 moves relative to the body 5802 a distance 5814 between the arms 5810, 5812 can change. For example, as the implant engagement head 5808 slides into the distal portion 5806 of the body 5802, the distance 5814 between the arms 5810, 5812 can decrease. Conversely, as the implant engagement head 5808 slides out of the distal portion 5806 of the body 5802, the distance 5814 between the arms 5810, 5812 can increase.

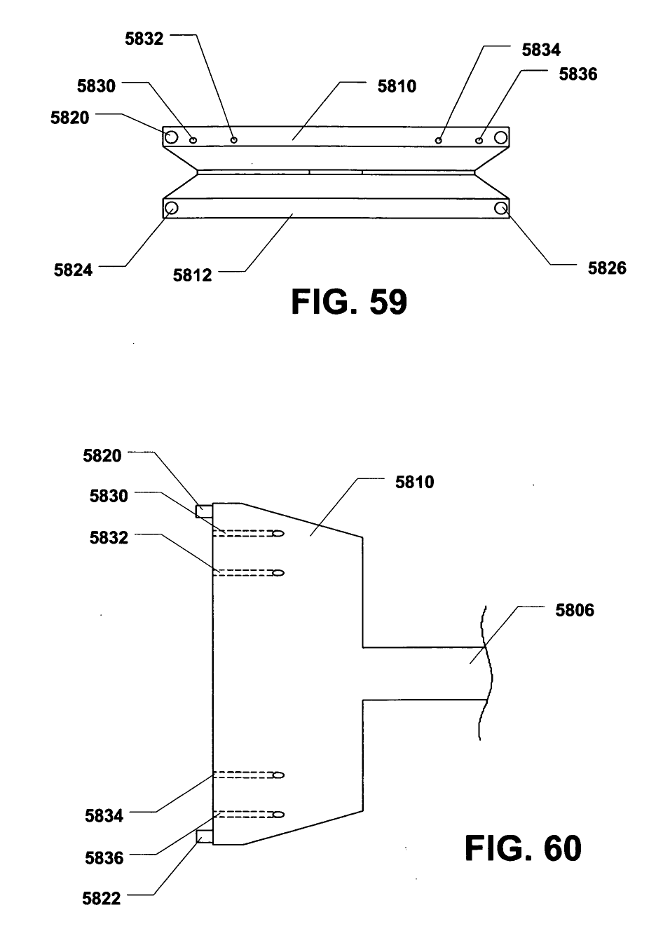

As illustrated in FIG. 59, the superior arm 5810 of the implant engagement head 5808 includes a first superior dowel 5820 and a second superior dowel 5822. The inferior arm 5812 of the implant engagement head 5808 includes a first inferior dowel 5824 and a second inferior dowel 5826. As shown, the dowels 5820, 5822, 5824, 5826 can be circular. Alternatively, the dowels 5820, 5822, 5824, 5826 can be triangular, square, rectangular, elliptical, or any polygonal shape.

FIG. 59 and FIG. 60 indicates that the superior arm 5810 of the implant engagement head 5808 can also include a first injector needle guide 5830, a second injector needle guide 5832, a third injector needle guide 5834, and a fourth injector needle guide 5836. As shown in FIG. 60, the injector needle guides 5830, 5832, 5834, 5836 extend through the implant engagement head 5808. During implantation of an intervertebral prosthetic disc, e.g., an intervertebral prosthetic disc according to one or more of the embodiments described herein, the injector needle guides 5830, 5832, 5834, 5836 can be used to properly align a material injector needle with a port established within the intervertebral prosthetic disc that is in fluid communication with an expandable motion limiter.

### CONCLUSION

With the configuration of structure described above, the intervertebral prosthetic disc according to one or more of the embodiments provides a device that may be implanted to replace a natural intervertebral disc that is diseased, degenerated, or otherwise damaged. The intervertebral prosthetic disc can be disposed within an intervertebral space between an inferior vertebra and a superior vertebra. Further, after a patient fully recovers from a surgery to implant the intervertebral prosthetic disc, the intervertebral prosthetic disc can provide relative motion between the inferior vertebra and the superior vertebra that closely replicates the motion provided by a natural intervertebral disc. Accordingly, the intervertebral prosthetic disc provides an alternative to a fusion device that can be implanted within the intervertebral space between the inferior vertebra and the superior vertebra to fuse the inferior vertebra and the superior vertebra and prevent relative motion there between.

During implantation, the surgeon can engage an intervertebral prosthetic disc with an implant inserter, e.g., an implant inserter according to one or more of the embodiments described herein, and use the implant inserter to implant the intervertebral prosthetic disc and inflate at least one expandable motion limiter incorporated into the intervertebral prosthetic disc. After the expandable motion limiter is inflated, the implant inserter can be disengaged from the intervertebral prosthetic implant and removed.

A surgeon may inflate the expandable motion limiter in order to limit the motion of a superior component with respect to an inferior component. As such, the surgeon can limit the motion of a superior vertebra with respect to an inferior vertebra. The flexibility to alter the range of motion of an intervertebral prosthetic device that is configured according to one or more of the embodiments disclosed herein can allow a surgeon to compensate for a deformity in the segment of the spinal column that includes, or is adjacent to, the superior vertebra and inferior vertebra in question. As such, a patient may be given a chance to recover from disc implant surgery with greater mobility than the mobility provided by a fusion device.

It can be appreciated that more than one of the features described above can be combined in another embodiment of an intervertebral prosthetic device. For example, one or more expandable motion limiters can extend from a superior component and one or more expandable motion limiters can extend from an inferior component. Each of the expandable motion limiters can be injected with material in order to limit the motion of the superior component with respect to the inferior component.

The above-disclosed subject matter is to be considered illustrative, and not restrictive, and the appended claims are intended to cover all such modifications, enhancements, and other embodiments that fall within the true spirit and scope of the present invention. For example, it is noted that the components in the exemplary embodiments described herein are referred to as “superior” and “inferior” for illustrative purposes only and that one or more of the features described as part of or attached to a respective half may be provided as part of or attached to the other half in addition or in the alternative. Thus, to the maximum extent allowed by law, the scope of the present invention is to be determined by the broadest permissible interpretation of the following claims and their equivalents, and shall not be restricted or limited by the foregoing detailed description.

1. An intervertebral prosthetic disc to be installed within an intervertebral space between adjacent first and second vertebrae, the intervertebral prosthetic disc comprising:
a first component configured to engage the first vertebra;
a second component configured to engage the second vertebra; and
an expandable motion limiter, wherein the expandable motion limiter is configured to move from a deflated position to an inflated position.

2. The intervertebral prosthetic disc of claim 1, wherein the expandable motion limiter is configured to limit relative motion between the first component and the second component when inflated.

3. The intervertebral prosthetic disc of claim 2, wherein the expandable motion limiter is inflated with an injectable extended use approved medical material.

4. The intervertebral prosthetic disc of claim 3, wherein the injectable extended use approved medical material comprises a polymer material.

5. The intervertebral prosthetic disc of claim 4, wherein the polymer material comprises a polyurethane material, a polyolefin material, a polyether material, a silicone material, or a combination thereof.

6. The intervertebral prosthetic disc of claim 5, wherein the polyolefin material comprises a polypropylene, polyethylene, halogenated polyolefin, flouropolyolefin, or a combination thereof.

7. The intervertebral prosthetic disc of claim 5, wherein the polyether material comprises polyetherketone (PEK), polyetheretherketone (PEEK), polyetherketoneketone (PEKK), polyaryletherketone (PAEK), or a combination thereof.

8. The intervertebral prosthetic disc of claim 5, wherein the silicon material comprises silicone hydrogel.

9. The intervertebral prosthetic disc of claim 3, wherein the injectable extended use approved medical material comprises a fluid.

10. The intervertebral prosthetic disc of claim 9, wherein the fluid comprises sterile water, saline, sterile air, or a combination thereof.

11. The intervertebral prosthetic disc of claim 2, wherein the first component comprises:
a support plate; and
an articular surface established on the support plate, wherein the expandable motion limiter is attached to the articular surface.

12. The intervertebral prosthetic disc of claim 11, wherein the first component further comprises a projection extending from the support plate and wherein the expandable motion limiter at least partially surrounds the projection.

13. The intervertebral prosthetic disc of claim 11, wherein the first component further comprises:
a fluid channel established within the support plate, wherein the fluid channel is configured to communicate with the expandable motion limiter; and
a port established within the support plate, wherein the port is configured to communicates with the fluid channel.

14. The intervertebral prosthetic disc of claim 13, wherein the port is configured to communicate the injectable extended use approved medical material to the fluid channel.

15. The intervertebral prosthetic disc of claim 13, wherein the fluid channel is configured to communicate the injectable extended use approved medical material to the expandable motion limiter.

16. The intervertebral prosthetic disc of claim 2, wherein the second component comprises:
a support plate; and
an articular surface established on the support plate, wherein the expandable motion limiter is attached to the articular surface.

17. The intervertebral prosthetic disc of claim 16, wherein the second component further comprises:
a fluid channel established within the support plate, wherein the fluid channel is configured to communicate with the expandable motion limiter; and
a port established within the support plate, wherein the port is configured to communicates with the fluid channel.

18. The intervertebral prosthetic disc of claim 17, further comprising an implant inserter engagement hole, wherein the port is established within the implant inserter engagement hole.

19. The intervertebral prosthetic disc of claim 3, wherein the injectable extended use approved medical material is a biological factor.

20. The intervertebral prosthetic disc of claim 19, wherein the biological factor includes fibroblasts, lipoblasts, chondroblasts, differentiated stem cells, or a combination thereof.

21. An intervertebral prosthetic disc to be installed within an intervertebral space between an inferior vertebra and a superior vertebra, the intervertebral prosthetic disc comprising:
an inferior component configured to engage the inferior vertebra; and
a superior component configured to engage the superior vertebra, the superior component comprising:
a superior support plate; and
an expandable motion limiter attached to the superior support plate, wherein the expandable motion limiter is configured to move from a deflated position to an inflated position.

22.-34. (canceled)

35. An intervertebral prosthetic disc to be installed within an intervertebral space between an inferior vertebra and a superior vertebra, the intervertebral prosthetic disc comprising:
a superior component configured to engage the inferior vertebra; and
an inferior component configured to engage the inferior vertebra, the inferior component comprising:
an inferior support plate; and
an expandable motion limiter attached to the inferior support plate, wherein the expandable motion limiter is configured to move from a deflated position to an inflated position.

36.-48. (canceled)

49. A method of installing an intervertebral prosthetic disc within an intervertebral space between an inferior vertebra and a superior vertebra of a patient, the method comprising:
implanting the intervertebral prosthetic disc within the intervertebral space; and
inflating an expandable motion limiter within the intervertebral prosthetic disc.

50. (canceled)

51. (canceled)

52. The method of claim 49, further comprising checking the relative motion between the inferior vertebra and the superior vertebra.

53.-61. (canceled)

62. An intervertebral prosthetic disc to be installed within an intervertebral space between an inferior vertebra and a superior vertebra, the intervertebral prosthetic disc comprising:
a superior component configured to engage the superior vertebra, the superior component comprising:
a superior support plate; and
an expandable motion limiter attached to the superior support plate, wherein the expandable motion limiter is configured to move from a deflated position to an inflated position, and
an inferior component configured to engage the inferior vertebra, the inferior component comprising:
an inferior support plate; and
an expandable motion limiter attached to the inferior support plate, wherein the expandable motion limiter is configured to move from a deflated position to an inflated position.
